(19) United States
(12) Patent Application Publication
(10) Pub. No.: US 2004/0148655 A1
(43) Pub. Date: Jul. 29, 2004

(54) Dwf5 mutants

(75) Inventors: Sunghwa Choe, Seoul (KR); Kenneth A. Feldmann, Newbury Park, CA (US)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: The Arizona Board of Regents on Behalf of the University of Arizona, an Arizona corporation

(21) Appl. No.: 10/793,373

(22) Filed: Mar. 4, 2004

Related U.S. Application Data
(62) Division of application No. 09/817,774, filed on Mar. 26, 2001.
(60) Provisional application No. 60/192,202, filed on Mar. 27, 2000.

(51) Int. Cl. A01H 1/00; C12N 15/82; C07H 21/04; C12N 9/24; C12N 5/04

(52) U.S. Cl. 800/287; 536/23.6; 435/419; 435/200

(57) ABSTRACT

Dwarf5 (dwf5) mutants and methods of using the same are disclosed. The dwf5 polynucleotides can be used in the production of transgenic plants which display at least one dwf5 phenotype, so that the resulting plants have altered structure or morphology. Also described is the DWF5 genomic sequence.

### CROSS-REFERENCE TO RELATED APPLICATION

This application is related to provisional patent application serial No. 60/192,202, filed Mar. 27, 2000, from which priority is claimed under 35 USC § 119(e)(1) and which is incorporated herein by reference in its entirety.

### TECHNICAL FIELD

The present invention relates generally to plants that display altered structure or morphology and to the genes imparting such pheontypes. In particular, the present invention pertains to Dwarf5 (dwf5) mutants and methods of using the same. The present invention also relates to isolated polynucleotides that encode regulatory regions of dwf5.

### BACKGROUND OF THE INVENTION

The brassinosteroids (BRs) are a group of plant steroid hormones that help regulate many different aspects of plant growth and development. BRs are known to stimulate cell elongation and division, and are also involved in vascular system differentiation, reproduction, and stress responses (Altmann, 1998; Clouse and Sasse, 1998). Recently, it has been shown that mutants defective either in BR biosynthesis or signaling, display altered developmental phenotypes including dwarfism, reduced fertility, and abnormal vasculature (Clouse and Feldmann, 1999).

BR dwarf mutants can be divided into two classes. The first class of mutants is perturbed in BR signaling. For example, Clouse, et al., (1996) isolated a signaling mutant, brassinosteroid insensitive1 (bri1), that was resistant to exogenously applied Brassinosteroids. BRI1 has been cloned and shown to encode a leucine-rich repeat receptor kinase, suggesting a role for BR perception at the cellular membrane (Li and Chory, 1997). Recently, it was demonstrated that bri1 mutants accumulate significant amounts of brassinolide (BL) and its precursors compared to wild-type controls, suggesting that perception is coupled to homeostasis of endogenous BR levels (Noguchi, et al., 1999b).

The other class of BR mutants includes a large number of dwarfs that are defective in BR biosynthesis. Plants produce Brassinosteroids using sterols as precursors, and the sterol biosynthetic pathway uses mevalonic acid as a precursor to synthesize sterols, such as sitosterol, stigmasterol, and campesterol. These sterols are modified by the BR-specific pathway to produce the end product, BL, and its congeners. Thus, mutants that are defective in either the sterol or BR-specific pathway display a typical BR dwarf phenotype, and can be rescued to a wild-type phenotype by exogenous application of Brassinosteroids.

de-etiolated2 (det2) and constitutive photomorphogenesis and dwarfism (cpd) were the pioneering mutants found to be defective in the BR-specific pathway. det2 mutants were shown to be disrupted in 5α-reductase activity (Fujioka, et al., 1997; Li, et al., 1996; Li, et al., 1997; Noguchi, et al., 1999a), and cpd was found to be due to a lesion in a cytochrome P450 (CYP90A) that is involved in the side chain hydroxylation at C-23 (Szekeres, et al., 1996). Furthermore, it has been shown that DWARF4 (DWF4) encodes a cytochrome P450 (CYP90B) that mediates the putative rate limiting C-22 hydroxylation step (Choe, et al., 1998). Another cytochrome P450 (CYP85) has been isolated from tomato and shown to be involved in the two consecutive steps of C-6 oxidation (Bishop, et al., 1996, 1999). Recently, Ephritikhine et al. (1999) reported that hypersensitive to abscisic acid and auxin1 (sax1) is defective in a step before DET2, but the SAX1 gene has not yet been identified.

The characteristic phenotype of BR dwarf mutants has been instrumental in isolating additional mutants, and their corresponding genes perturbed in the complex plant sterol biosynthesis network. dwf1 was the first mutant isolated to have this dwarf phenotype (Feldmann, et al., 1989). We and others have shown that dwf1 is defective in C-24 reduction, and that DWF1 encodes a FAD-binding oxidoreductase (Choe, et al., 1999a; Klahre, et al., 1998; Takahashi, et al., 1995). The pea lkb mutant is deficient in the same reaction as Arabidopsis dwf1 (Nomura, et al., 1999). Another sterol mutant, Arabidopsis dwf7/ste1, has been isolated and found to be defective in the Δ⁷ sterol C-5 desaturase gene (Gachotte, et al., 1995; 1996, Husselstein, et al., 1999, Choe, et al., 1999b).

The enzymatic step between DWF7 and DWF1 is mediated by a sterol Δ⁷ reductase (S7R). This enzyme was previously identified in rat and maize (Taton and Rahier, 1991). The gene for this enzyme has been cloned by heterologous expression of an Arabidopsis cDNA library in yeast, and subsequent selection of transformants resistant to the polyene fungicide nystatin (Lecain, et al., 1996). Nystatin is specifically toxic to yeast because it acts on yeast ergosterol containing Δ^{5,7}-dienic double bonds. However, transformants containing an Arabidopsis Δ⁷ reductase gene could saturate the double bond at C-7, and become resistant to the fungicide.

S7R activity is essential for growth in humans. Children deficient in this enzyme are afflicted with Smith-Lemli-Opitz syndrome (SLOS). Symptoms of this congenital anomaly include microcephaly with mental retardation and hypertonicity as well as malformation of many different organs (Smith, et al., 1964). The patients have elevated levels of 7-dehydrocholesterol, which is a precursor of the S7R reaction (Salen, et al., 1996). Using information obtained from the Arabidopsis S7R cDNA, three independent groups cloned the human 7-dehydrocholesterol reductase gene (DHCR7) (Moebius, et al., 1998; Wassif, et al., 1998; Waterham, et al., 1998). Sequencing of alleles collected from independent SLOS patients revealed that their DHCR7 genes encode malfunctional proteins (Fitzky, et al., 1998; Wassif, et al., 1998).

Currently, little is known about the downstream events that occur in response to signals in the above pathways that ultimately control cell size. This is because the biochemical and cell biological processes involved have thus far been difficult to address. In addition, there is little information about the integration of regulatory signals converging at the cell from different signaling pathways and the ways they are coordinately controlled. In particular, the interaction of light and hormones in the control of cell elongation is not clear. Thus, there remains a need for the identification and characterization of additional mutants and polypeptides encoded thereby involved in these pathways of plant growth.

### SUMMARY OF THE INVENTION

The present invention is based on the discovery of various mutants of a BR biosynthetic locus, designated dwarfs (dwf5). dwf5 mutants are defective in a step mediated by an S7R. At least six independent alleles have been characterized herein, each of which contain loss-of-function mutations in the sterol Δ⁷ reductase gene. These include a putative mRNA instability mutation in dwf5-1,3′ and 5′ splice site mutations in dwf5-2 and dwf5-6, respectively, premature stop codons in dwf5-3 (R400Z) and dwf5-5 (R409Z), and a missense mutation in dwf5-4 (D257N) that causes an amino acid substitution. Similar to humans deficient for DHCR7, mutations in this gene result in severely altered growth and development in plants.

Accordingly, in one embodiment, the present invention is directed to an isolated polynucleotide selected from the group consisting of (a) a dwf5-1 polynucleotide comprising the dwf5-1 nucleotide sequence depicted in FIG. 7; (b) a dwf5-2 polynucleotide comprising the dwf5-2 nucleotide sequence depicted in FIG. 7; (c) a dwf5-3 polynucleotide comprising the dwf5-3 nucleotide sequence depicted in FIG. 7; (d) a dwf5-4 polynucleotide comprising the dwf5-4 nucleotide sequence depicted in FIG. 7; (e) a dwf5-S polynucleotide comprising the dwf5-5 nucleotide sequence depicted in FIG. 7; (f) a dwf5-6 polynucleotide comprising the dwf5-6 nucleotide sequence depicted in FIG. 7; (g) a DWF5 polynucleotide comprising the genomic DWF5 sequence depicted in FIG. 7; (h) a polynucleotide comprising a nucleotide sequence having at least about 50% sequence identity to the nucleotide sequence of (a), (b), (c), (d), (e), (f) or (g); (i) a fragment of (a), (b), (c), (d), (e), (f), (g) or (h) comprising at least about 15 contiguous nucleotides therefrom; and (j) complements or reverse complements of (a), (b), (c), (d), (e), (f), (g), (h) or (i). In certain embodiments, the isolated polynucleotide imparts at least one dwf5 mutant phenotype when expressed in a plant.

In a further embodiment, the invention is directed to a recombinant vector comprising (i) a polynucleotide as described above; and (ii) control elements operably linked to said polynucleotide whereby a coding sequence within said polynucleotide can be transcribed and translated in a host cell, as well as host cells comprising the vector.

In another embodiment, the invention is directed to an isolated polynucleotide selected from the group consisting of (a) a polynucleotide comprising a nucleotide sequence having at least about 50% sequence identity to the genomic DWF5 sequence depicted in FIG. 7; (b) a fragment of (a) comprising at least about 15 contiguous nucleotides therefrom; and (c) complements or reverse complements of (a) or (b). Recombinant vectors comprising the polynucleotide, as well as host cells transformed therewith, are also provided.

In still a further embodiment, the subject invention is directed to an isolated polynucleotide selected from the group consisting of (a) a polynucleotide comprising the nucleotide sequence depicted at nucleotide positions 1-633 of FIG. 7; (b) a polynucleotide comprising the nucleotide sequence depicted at nucleotide positions 634-670 of FIG. 7; (c) a polynucleotide comprising the nucleotide sequence depicted at nucleotide positions 4045-4243 of FIG. 7; (d) a polynucleotide comprising an intron sequence as depicted in FIG. 7; (e) a polynucleotide comprising a nucleotide sequence having at least about 50% sequence identity to the nucleotide sequence of (a), (b), (c) or (d); (f) a fragment of (a), (b), (c), (d) or (e) comprising at least about 15 contiguous nucleotides therefrom; and (g) complements or reverse complements of (a), (b), (c), (d), (e) or (f).

In another embodiment, the invention is directed to a recombinant vector comprising: (i) a polynucleotide as specified above, which includes a DWF5 control element; and (ii) a heterologous coding sequence operably linked to said polynucleotide. Also provided are host cells transformed with the vector.

In still further embodiments, the invention is directed to a method of producing a recombinant polypeptide comprising:

(a) providing a host cell as specified above; and

(b) culturing said host cell under conditions whereby a recombinant polypeptide encoded by the coding sequence present in said recombinant vector is expressed.

In another embodiment, the invention is directed to a transgenic plant comprising a polynucleotide as described above.

In still another embodiment, the invention is directed to a method of producing a transgenic plant comprising:

(a) introducing a polynucleotide or recombinant vector as described above into a plant cell to produce a transformed plant cell; and

(b) producing a transgenic plant from the transformed plant cell.

In another embodiment, the invention is directed to a method for producing a transgenic plant having an altered phenotype relative to the corresponding wild-type plant comprising:

(a) introducing a polynucleotide or recombinant vector as described above into a plant cell; and

(b) producing a transgenic plant from the plant cell, said transgenic plant having an altered phenotype relative to the corresponding wild-type plant.

In certain embodiments, the polynucleotide is operably linked to a promoter selected from the group consisting of a tissue-specific promoter, an inducible promoter and a constitutive promoter. Additionally, the polynucleotide may be overexpressed or may inhibit expression of dwf5. Additionally, the method may be one wherein at least first and second polynucleotides are introduced into the plant cell, said first and second polynucleotides operably linked to at least first and second tissue-specific promoters, wherein said first polynucleotide is overexpressed and said second polynucleotide inhibits expression of dwf5.

In still further embodiments, the invention is directed to a method of modulating an endogenous DWF5 polypeptide in a transgenic plant comprising providing a polynucleotide as described above. The polynucleotide may be overexpressed or expression of the polynucleotide may be inhibited.

In another embodiment the invention is directed a method for altering the biochemical activity of a cell comprising:

(a) introducing at least one polynucleotide as described above into the cell; and

(b) causing expression of said polynucleotide such that the biochemical activity of the cell is altered. The polynucleotide may be introduced into the cell ex vivo or in vivo.

In certain embodiments, the biochemical activity is selected from the group consisting of altered sterol Δ⁷ reductase activity and altered sterol composition. Additionally, more than one dwf5 polynucleotide may provided to the cell.

In yet a further embodiment, the subject invention is directed to a method for regulating the cell cycle of a plant cell comprising:

(a) providing a polynucleotide as described above to a plant cell; and

(b) expressing the polynucleotide to provide a DWF5 polypeptide, wherein the DWF5 polypeptide is provided in amounts such that cell cycling is regulated. The plant cell may be provided in vitro and cultured under conditions suitable for expressing the DWF5 polypeptide. Alternatively, the polynucleotide may be provided in vivo.

In another embodiment, the invention is directed to a method of modulating mRNA levels in a plant cell comprising:

(a) providing a plant cell; and

(b) introducing a recombinant vector as described above into the plant cell to produce a transformed plant cell. The recombinant vector may be introduced into the plant cell in vitro or in vivo.

In certain embodiments, the plant cell is from a plant tissue selected from the group consisting of the shoot apex and unopened flower (SAF), stem, mature silique, pedicel, rosette leaf, root, dark-grown seedling and callus.

In still a further embodiment, the subject invention is directed to a chimeric polypeptide comprising a first amino acid sequence of a DWF5 polypeptide and a second amino acid sequence of a heterologous polypeptide.

These and other embodiments of the present invention will readily occur to those of ordinary skill in the art in view of the disclosure herein.

### BRIEF DESCRIPTION OF THE FIGURES

FIG. 1 presents a comparison of the phenotypes of Ws-2 wild-type and dwf5-1 seeds.

FIG. 2 presents data showing the biochemical complementation of dwf5-1 plants with brassinolide (BL) and its biosynthetic intermediates.

FIG. 3 presents the results of biochemical analyses of metabolism of ¹³C-labeled mevalonic acid (MVA) and endogenous Brassinosteroid levels.

FIG. 4A presents a schematic representation of the dwf5 locus. FIG. 4B presents a schematic view of the dwf5 region from exon 9 to the 3′ UTR.

FIG. 5 presents multiple sequence alignments of sequences with some similarities to DWF5.

FIGS. 6A and 6B are representations of the results of RNA blot analysis (Northerns) of DWF5 transcripts.

FIG. 7 presents the genomic sequence of the DWF5 locus including annotations regarding the transcription start site (nucleotide 634), translation start site (nucleotide 671), exons (those portions of the sequences with corresponding polypeptide sequence) and introns (no corresponding polypeptide sequence indicated), sites of mutations corresponding to dwf5-4, dwf5-2, dwf5-3, dwf5-5, dwf5-6, dwf5-1, and translation products resulting from mis-spliced mRNAs.

FIG. 8 shows a sequence of a DWF5 cDNA and the corresponding translation product.

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., Evans, et al., Handbook of Plant Cell Culture (1983, Macmillan Publishing Co.); Binding, Regeneration of Plants, Plant Protoplasts (1985, CRC Press); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a polypeptide” includes a mixture of two or more polypeptides, and the like.

The following amino acid abbreviations are used throughout the text:

Alanine: Ala (A) Arginine: Arg (R)

Asparagine: Asn (N) Aspartic acid: Asp (D)

Cysteine: Cys (C) Glutamine: Gln (O)

Glutamic acid: Glu (E) Glycine: Gly (G)

Histidine: His (H) Isoleucine: Ile (I)

Leucine: Leu (L) Lysine: Lys (K)

Methionine: Met (M) Phenylalanine: Phe (F)

Proline: Pro (P) Serine: Ser (S)

Threonine: Thr (T) Tryptophan: Trp (W)

Tyrosine: Tyr (Y) Valine: Val (V)

I. Definitions

In describing the present invention, the following terms will be employed, and are intended to be defined as indicated below.

The terms “nucleic acid molecule” and “polynucleotide” are used interchangeably and refer to a polymeric form of nucleotides of any length, either deoxyribonucleotides or ribonucleotides, or analogs thereof. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides may have any three-dimensional structure, and may perform any function, known or unknown. Nonlimiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D. C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, the degree of sequence similarity between polynucleotides can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 70%-85%, preferably at least about 85%-90%, more preferably at least about 90%-95%, and most preferably at least about 95%-98% sequence identity over a defined length of the molecules, or any percentage between the above-specified ranges, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

A “gene” as used in the context of the present invention is a sequence of nucleotides in a genetic nucleic acid (chromosome, plasmid, etc.) with which a genetic function is associated. A gene is a hereditary unit, for example of an organism, comprising a polynucleotide sequence that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids, wherein the gene does not encode an expressed product. Typically, a gene includes coding sequences, such as, polypeptide encoding sequences, and non-coding sequences, such as, promoter sequences, polyadenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′(amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence. “Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), translation enhancing sequences, and translation termination sequences. Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

A control element, such as a promoter, “directs the transcription” of a coding sequence in a cell when RNA polymerase will bind the promoter and transcribe the coding sequence into mRNA, which is then translated into the polypeptide encoded by the coding sequence.

“Expression enhancing sequences” typically refer to control elements that improve transcription or translation of a polynucleotide relative to the expression level in the absence of such control elements (for example, promoters, promoter enhancers, enhancer elements, and translational enhancers (e.g., Shine and Delagarno sequences).

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A “heterologous sequence” as used herein typically refers to a nucleic acid sequence that is not normally found in the cell or organism of interest. For example, a DNA sequence encoding a polypeptide can be obtained from a plant cell and introduced into a bacterial cell. In this case the plant DNA sequence is “heterologous” to the native DNA of the bacterial cell.

The “native sequence” or “wild-type sequence” of a gene is the polynucleotide sequence that comprises the genetic locus corresponding to the gene, e.g., all regulatory and open-reading frame coding sequences required for expression of a completely functional gene product as they are present in the wild-type genome of an organism. The native sequence of a gene can include, for example, transcriptional promoter sequences, translation enhancing sequences, introns, exons, and poly-A processing signal sites. It is noted that in the general population, wild-type genes may include multiple prevalent versions that contain alterations in sequence relative to each other and yet do not cause a discernible pathological effect. These variations are designated “polymorphisms” or “allelic variations.”

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

By “vector” is meant any genetic element, such as a plasmid, phage, transposon, cosmid, chromosome, virus etc., which is capable of transferring gene sequences to target cells. Generally, a vector is capable of replication when associated with the proper control elements. Thus, the term includes cloning and expression vehicles, as well as viral vectors and integrating vectors.

As used herein, the term “expression cassette” refers to a molecule comprising at least one coding sequence operably linked to a control sequence which includes all nucleotide sequences required for the transcription of cloned copies of the coding sequence and the translation of the mRNAs in an appropriate host cell. Such expression cassettes can be used to express eukaryotic genes in a variety of hosts such as bacteria, blue-green algae, plant cells, yeast cells, insect cells and animal cells. Under the invention, expression cassettes can include, but are not limited to, cloning vectors, specifically designed plasmids, viruses or virus particles. The cassettes may further include an origin of replication for autonomous replication in host cells, selectable markers, various restriction sites, a potential for high copy number and strong promoters.

A cell has been “transformed” by an exogenous polynucleotide when the polynucleotide has been introduced inside the cell. The exogenous polynucleotide may or may not be integrated (covalently linked) into chromosomal DNA making up the genome of the cell. In procaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cells containing the exogenous DNA.

“Recombinant host cells,” “host cells,” “cells,” “cell lines,” “cell cultures,” and other such terms denoting procaryotic microorganisms or eucaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

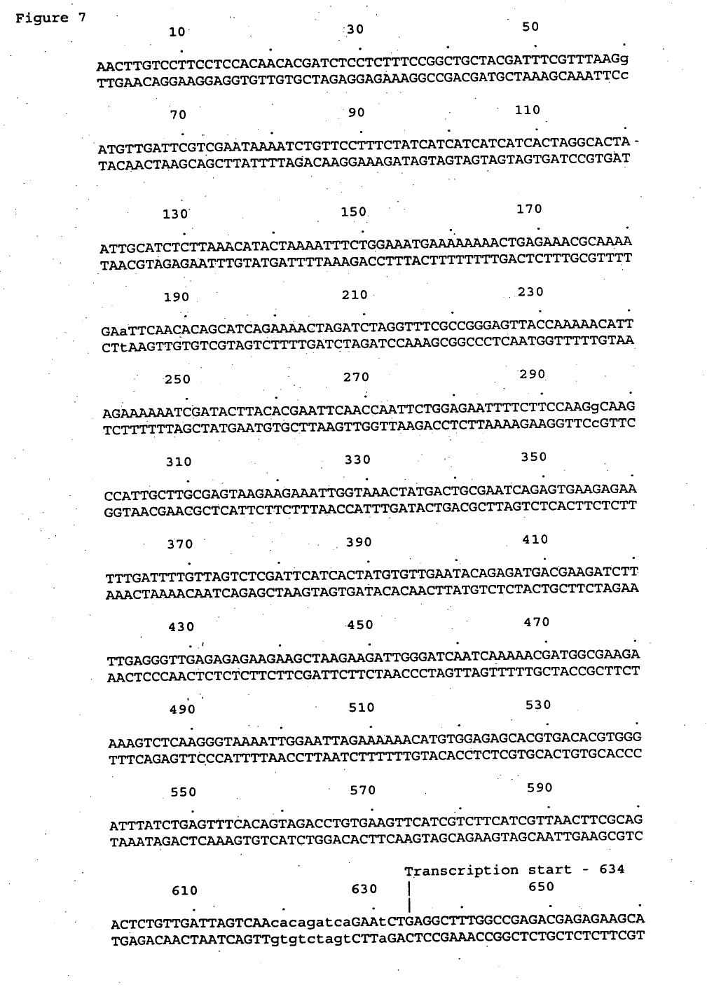

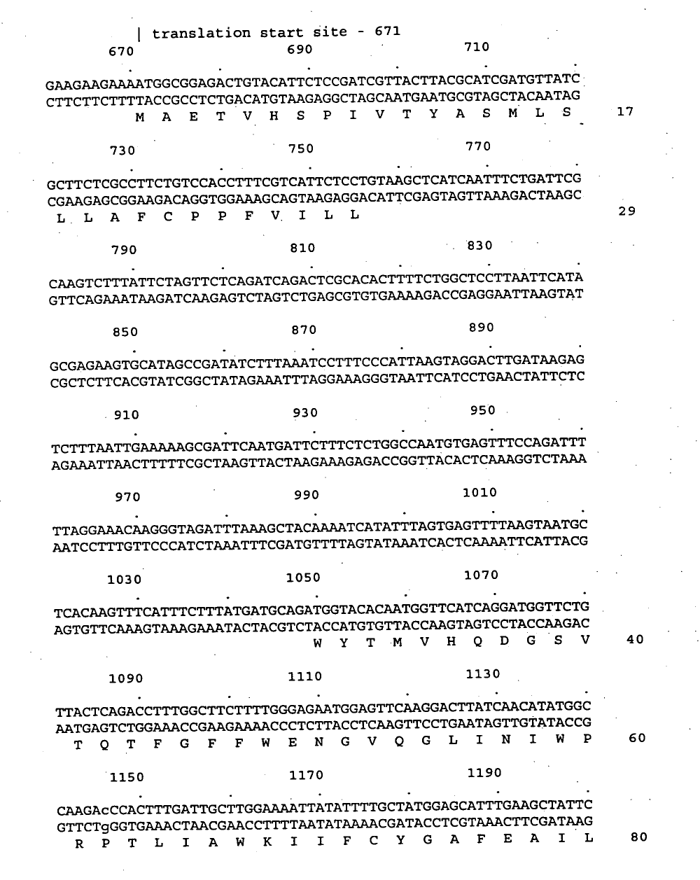

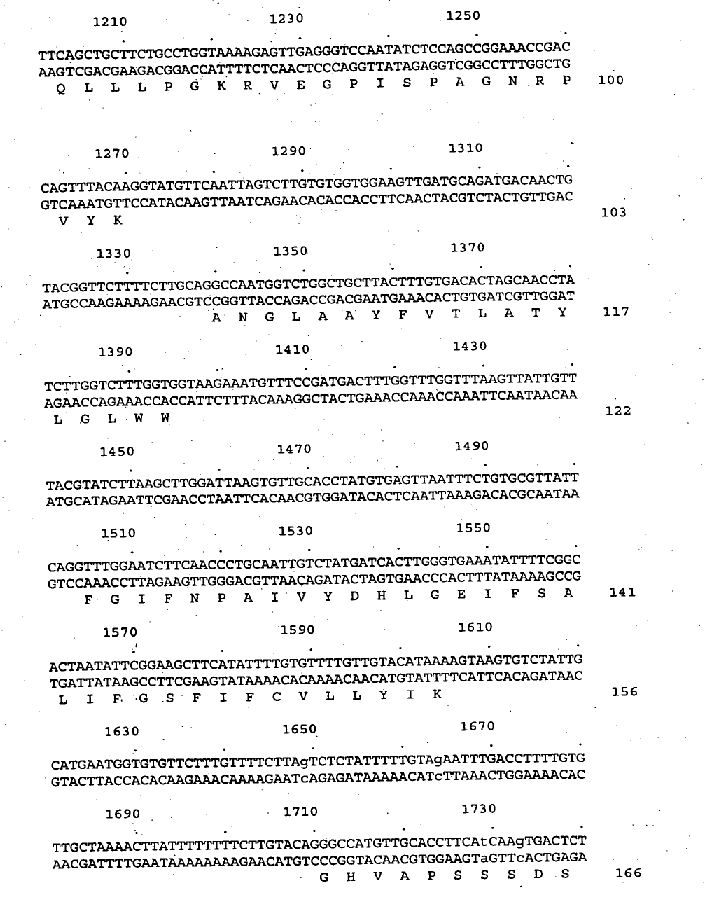

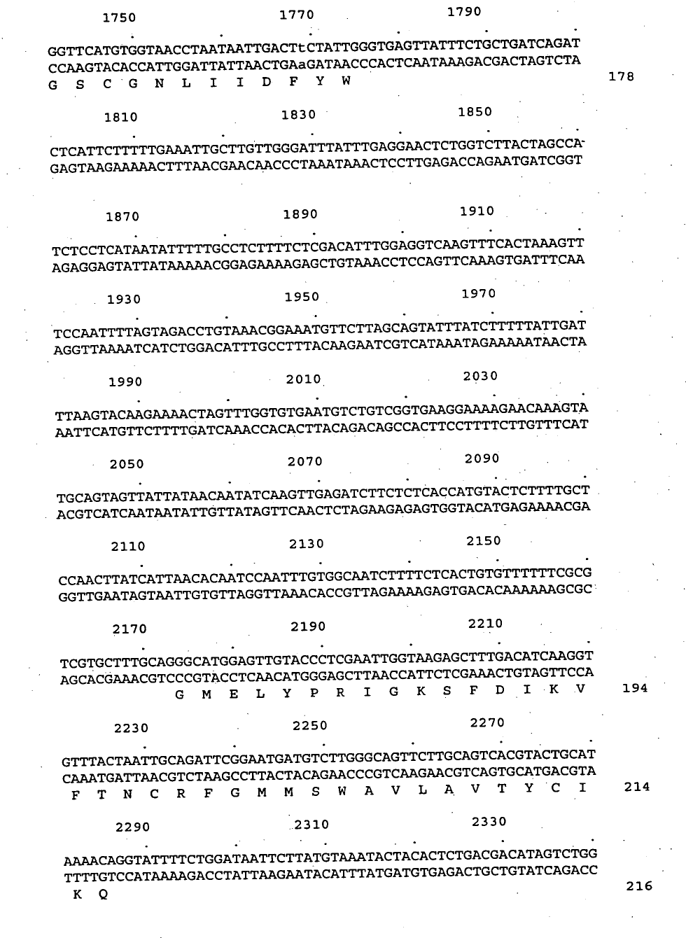

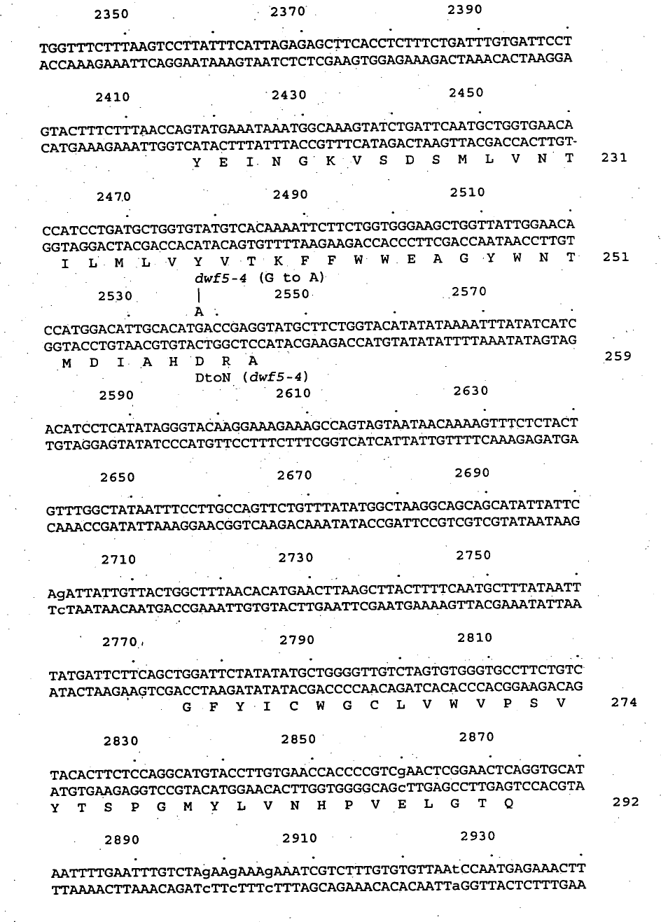

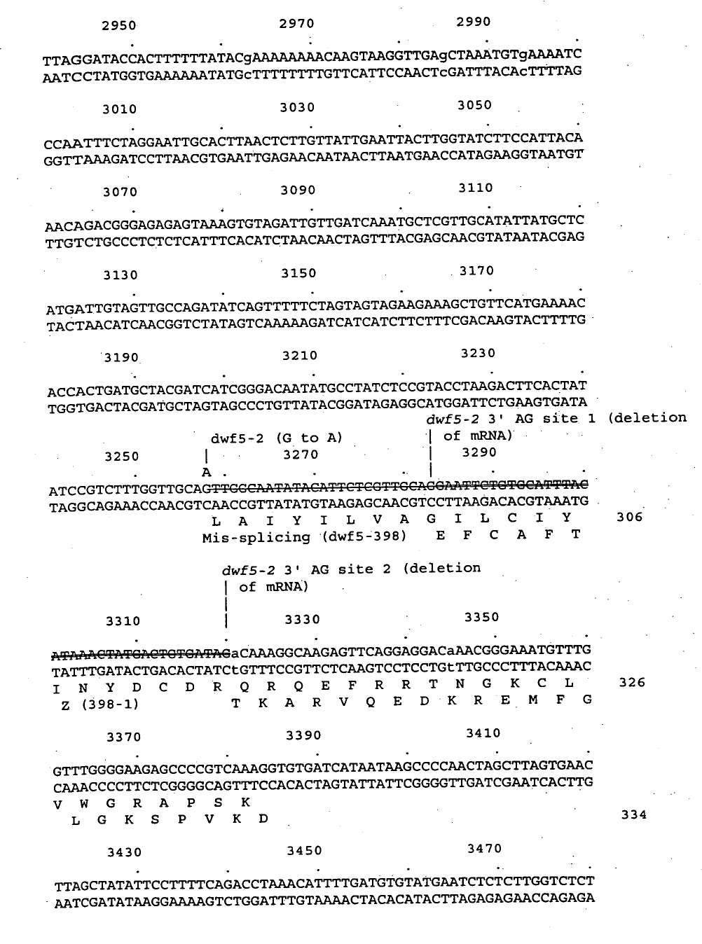

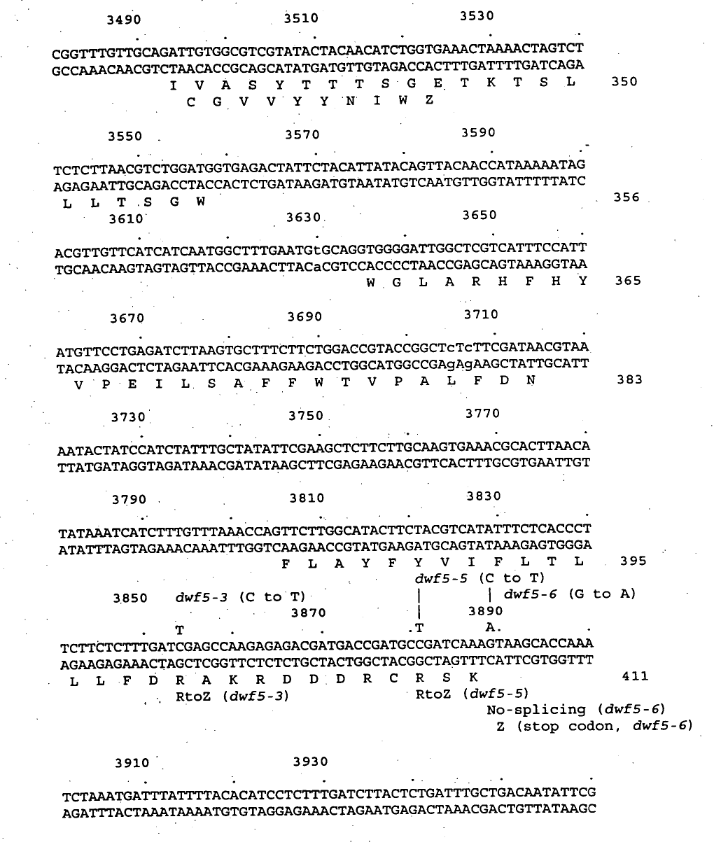

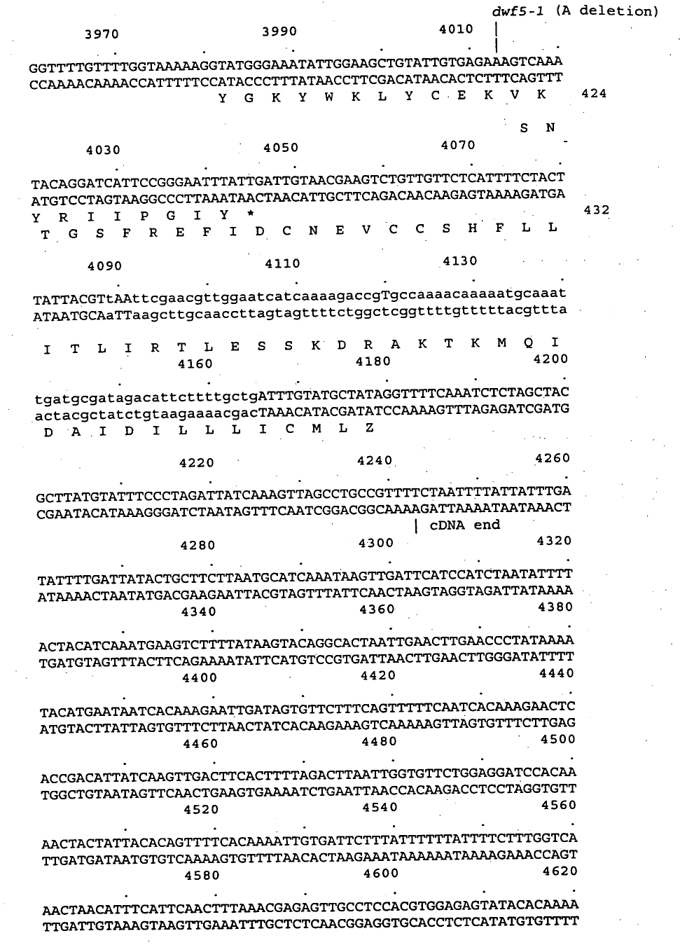

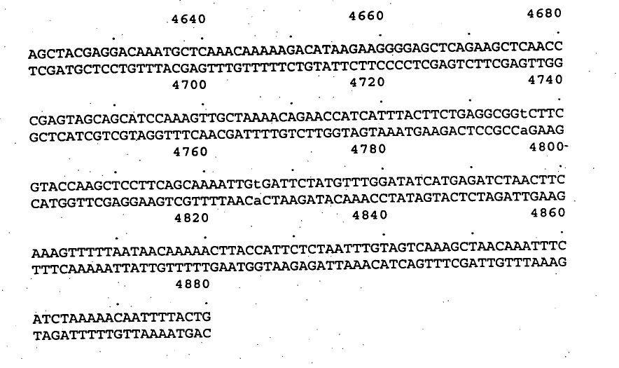

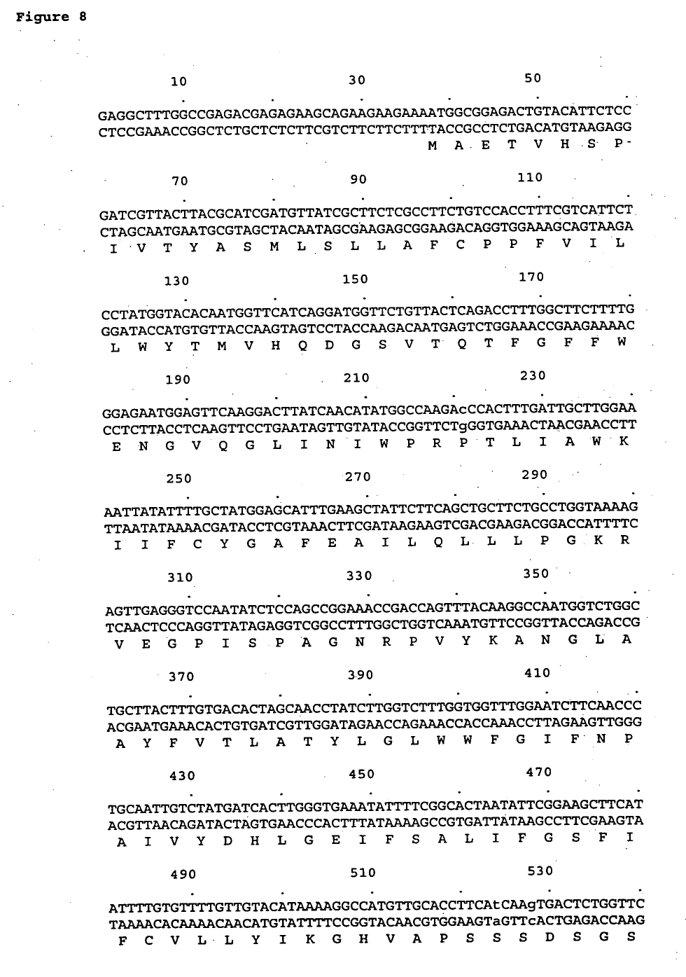

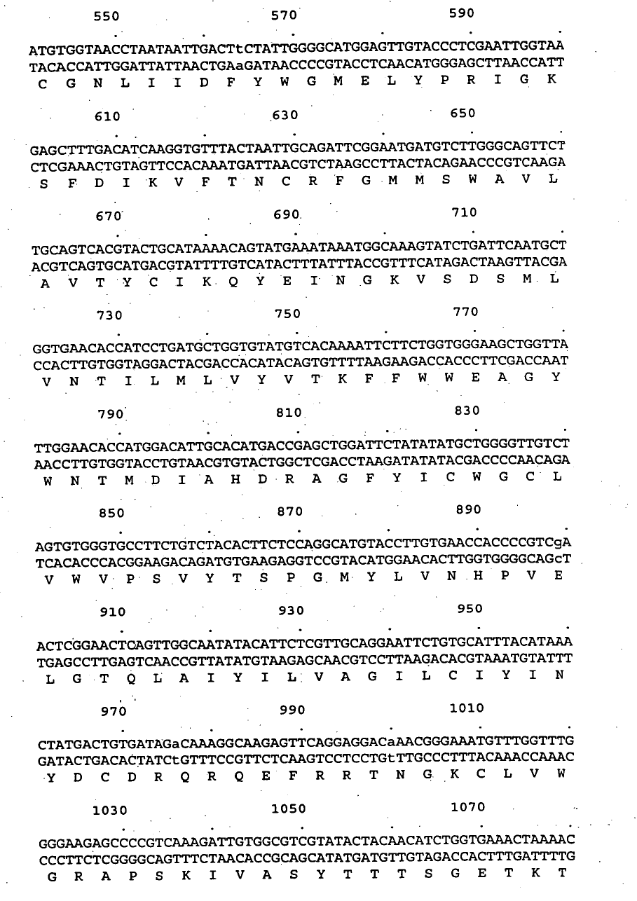

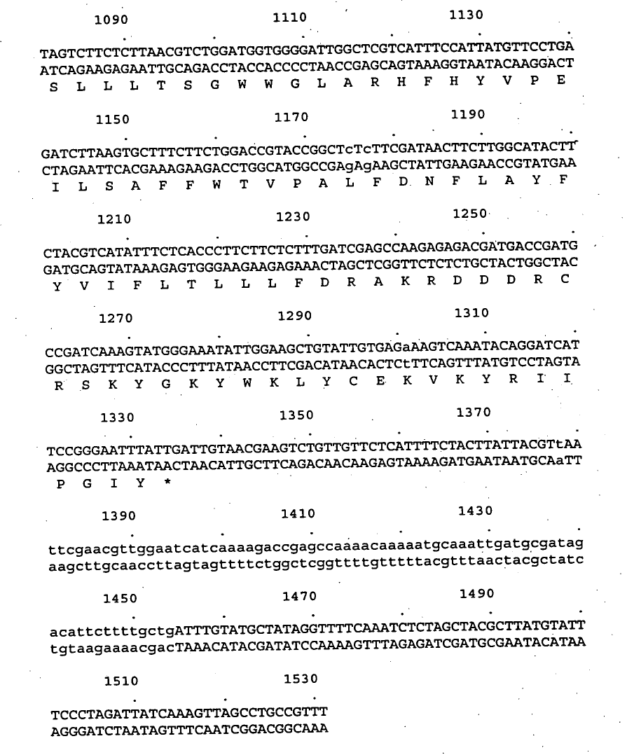

The term “DWF5 polynucleotide” refers to a polynucleotide derived from the DWF5 genomic sequence. The gene encodes a sterol Δ⁷ reductase that functions in the brassinolide (BL) biosynthetic pathway from squalene to BL and is also known as S7R. The S7R cDNA sequence and corresponding amino acid sequence are known and have been described in, e.g., Lecain et al. 1996 and GenBank accession number U49398. See, also, FIG. 8 herein showing the DWF5 cDNA sequence and corresponding amino acid sequence. The coding sequence spans nucleotide positions 38-1331 of FIG. 8, which corresponds to amino acid positions 1-432 of the figure. The 5′ UTR is shown at nucleotide positions 1-37 of FIG. 8 and the 3′ UTR is present at nucleotide positions 1332-1531 of the figure. FIG. 7 depicts the DWF5 genomic sequence and the corresponding amino acid sequence. As shown in FIG. 7, the genomic sequence spans the region from nucleotide positions 1-4940; the promoter region is present within nucleotide positions 1-633; the 5′ UTR is found at positions 634-670; the coding region begins at nucleotide position 671; the downstream 3′ UTR is present at nucleotide positions 4045-4243. Fragments of the sequence, as well as polynucleotide sequences homologous thereto, as defined above, and chimeras thereof, are captured by this definition. Thus, for example, a portion of the cDNA sequence shown in FIG. 8, linked to a single intron or portion of an intron from the genomic sequence, as shown in FIG. 7, is encompassed herein.

The term “dwf5 polynucleotide” as used herein encompasses a polynucleotide derived from the sequence depicted in FIGS. 7 and 8, as well as modifications, fragments and chimeras thereof. The term therefore encompasses alterations to the polynucleotide sequence, so long as the alteration results in a plant displaying one or more dwf5 phenotypic traits (described below) when the polynucleotide is expressed in a plant. Such modifications typically include deletions, additions and substitutions, to the native DWF5 sequence, so long as the mutation results in a plant displaying a dwf5 phenotype as defined below. These modifications may be deliberate, as through site-directed mutagenesis, or may be accidental, such as through mutations of plants which express the dwf5 polynucleotide or errors due to PCR amplification. The term encompasses expressed allelic variants of the wild-type dwf5 sequence which may occur by normal genetic variation or are produced by genetic engineering methods and which result in a detectable change in the wild-type dwf5 phenotype. Six particular dwf5 allelic variants described herein are dwf5-l through dwf5-6. These variants are discussed in detail below.

The term “dwf5 phenotype” as used herein refers to any microscopic or macroscopic change in structure or morphology of a plant, such as a transgenic plant, as well as biochemical differences, which are characteristic of a dwf5 plant, as compared to a progenitor, wild-type plant cultivated under the same conditions. For example, biochemically, dwf5 pedicels are converted to the wild-type length with the application of BL. Biosynthetic intermediates of BL, such as 6-deoxocathasterone (6-DeoxoCT), 22-hydroxycampesterol (22-OHCR), significantly increase dwf5 pedicel length. See, FIG. 2. Generally, morphological differences include a short robust stature, short internodes, an increased number of inflorescences, and small dark-green, round leaves similar to the phenotype previously reported for the other BR dwarfs (Azpiroz, et al., 1998; Choe, et al., 1999a, 1999b). The height of such plants will typically be 75% or less of the wild-type plant, more typically 50% or less of the wild-type plant, and even more typically 25% or less of the wild-type plant, or any integer in between. The morphological and biochemical differences described herein may arise from inhibition of transcription or translation, or from protein activity.

Thus, by dwf5 phenotype is meant an increase or decrease in any of the following activities which are associated with the presence or absence of a dwf5 polynucleotide or polypeptide sequence: brassinosteroid biosynthesis; sterol Δ⁷ reductase (S7R) activity; sterol composition; cell cycle; cell elongation and division; seed number; seed size; seed viability; organ size such as silique length, internodes, leaves, stems, pedicels and petioles; and increased number of inflorescences. Additional phenotypic morphological attributes of the dwf5 mutant are summarized in Table 1 of the examples.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein. Full-length proteins, analogs, and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular polypeptide may be obtained as an acidic or basic salt, or in neutral form. A polypeptide may be obtained directly from the source organism, or may be recombinantly or synthetically produced (see further below).

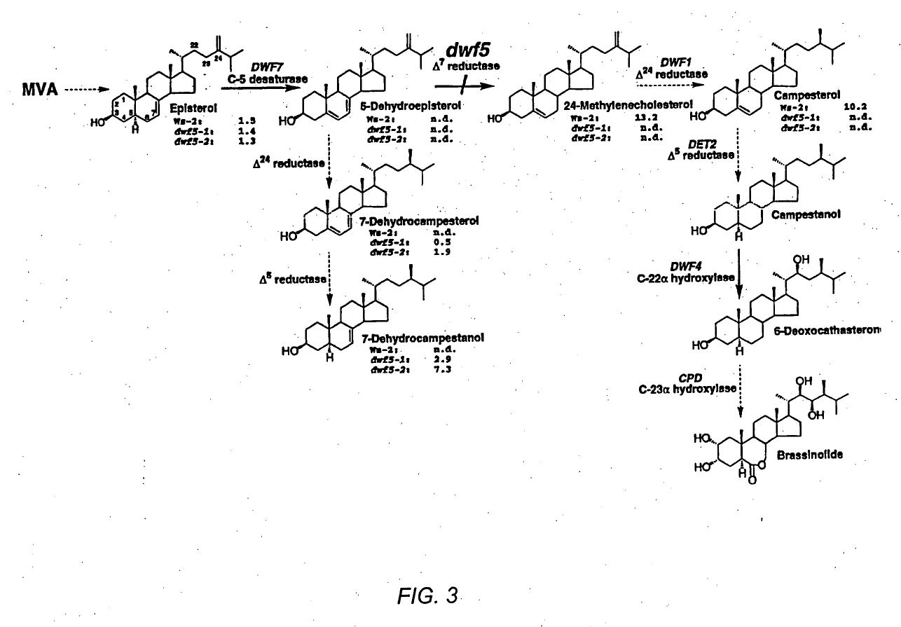

A “DWF5” polypeptide is a polypeptide as defined above, which is derived from a sterol Δ⁷ reductase that functions in the brassinolide (BL) biosynthetic pathway from squalene to BL (see, FIG. 3). The native sequence of full-length DWF5 is shown in FIG. 8. However, the term encompasses analogs and fragments of the native sequence so long as the protein functions for its intended purpose.

The term “DWF5 analog” refers to derivatives of DWF5, or fragments of such derivatives, that retain desired function, e.g., as measured in assays as described further below. In general, the term “analog” refers to compounds having a native polypeptide sequence and structure with one or more amino acid additions, substitutions (generally conservative in nature) and/or deletions, relative to the native molecule, so long as the modifications do not destroy desired activity. Preferably, the analog has at least the same activity as the native molecule. Methods for making polypeptide analogs are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. It is to be understood that the terms include the various sequence polymorphisms that exist, wherein amino acid substitutions in the protein sequence do not affect the essential functions of the protein.

By “purified” and “isolated” is meant, when referring to a polypeptide or polynucleotide, that the molecule is separate and discrete from the whole organism with which the molecule is found in nature; or devoid, in whole or part, of sequences normally associated with it in nature; or a sequence, as it exists in nature, but having heterologous sequences (as defined below) in association therewith. It is to be understood that the term “isolated” with reference to a polynucleotide intends that the polynucleotide is separate and discrete from the chromosome from which the polynucleotide may derive. The term “purified” as used herein preferably means at least 75% by weight, more preferably at least 85% by weight, more preferably still at least 95% by weight, and most preferably at least 98% by weight, of biological macromolecules of the same type are present. An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

By “fragment” is intended a polypeptide or polynucleotide consisting of only a part of the intact sequence and structure of the reference polypeptide or polynucleotide, respectively. The fragment can include a 3′ or C-terminal deletion or a 5′ or N-terminal deletion, or even an internal deletion, of the native molecule. A polynucleotide fragment of a dwf5 sequence will generally include at least about 15 contiguous bases of the molecule in question, more preferably 18-25 contiguous bases, even more preferably 30-50 or more contiguous bases of the dwf5 molecule, or any integer between 15 bases and the full-length sequence of the molecule. Fragments which provide at least one dwf5 phenotype as defined above are useful in the production of transgenic plants. Fragments are also useful as oligonucleotide probes, to find additional dwf5 sequences.

Similarly, a polypeptide fragment of a DWF5 molecule will generally include at least about 10 contiguous amino acid residues of the full-length molecule, preferably at least about 15-25 contiguous amino acid residues of the full-length molecule, and most preferably at least about 20-50 or more contiguous amino acid residues of the full-length DWF5 molecule, or any integer between 10 amino acids and the full-length sequence of the molecule. Such fragments are useful for the production of antibodies and the like.

By “transgenic plant” is meant a plant into which one or more exogenous polynucleotides has been introduced. Examples of means by which this can be accomplished are described below, and include Agrobacterium-mediated transformation, biolistic methods, electroporation, and the like. In the context of the present invention, the transgenic plant contains a polynucleotide which is not normally present in the corresponding wild-type plant and which confers at least one dwf5 phenotypic trait to the plant. The transgenic plant therefore exhibits altered structure, morphology or biochemistry as compared with a progenitor plant which does not contain the transgene, when the transgenic plant and the progenitor plant are cultivated under similar or equivalent growth conditions. Such a plant containing the exogenous polynucleotide is referred to here as an R₁ generation transgenic plant. Transgenic plants may also arise from sexual cross or by selfing of transgenic plants into which exogenous polynucleotides have been introduced. Such a plant containing the exogenous nucleic acid is also referred to here as an R₁ generation transgenic plant. Transgenic plants which arise from a sexual cross with another parent line or by selfing are “descendants or the progeny” of a R₁ plant and are generally called Fₙ plants or Sₙ plants, respectively, n meaning the number of generations.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Brief Description

The inventors herein have biochemically characterized the dwf5 mutant, elucidated the DWF5 genomic sequence and characterized at least six allelic variants of dwf5. As discussed further below, dwf5 mutants display multiple perturbations in growth and development. The primary cause of the dwarf morphology in dwf5 is a deficiency in Brassinosteroids. This conclusion is based on two lines of evidence. First, the dwarf phenotype in pedicels and inflorescences can be rescued to wild-type size by exogenous application of Brassinosteroids. Second, and more specifically, significantly reduced levels of sterols and Brassinosteroids are found in mutant tissues. Tracing the metabolism of ¹³C-labeled intermediates in dwf5 shows that dwf5 is deficient in an S7R. Loss-of-function mutations in the S7R are seen in six independent dwf5 alleles described herein.

As shown in the examples, dwf5 plants show growth defects in several organs as compared to wild-type plants, including short siliques and reduced seed set. While dwf5 plants have siliques with more seeds than other BR dwarfs, the seeds are aberrantly shaped, and do not germinate well. Treatment with BR increases the seed germination rate. Thus, without being bound by a particular theory, it may be that endogenous sterols or Brassinosteroids are required for seed development or for maintaining the viability of seeds. In support of a role for Brassinosteroids in seed maturation and dormancy, it has been reported that BR levels are high in pollen and seeds (reviewed in Altmann, 1998).

In contrast to all other BR dwarfs in Arabidopsis, the life cycle of dwf5-1 mutants is not significantly prolonged compared to that of wild type. This supports the idea that increased sterility in BR dwarf mutants is correlated with life span (Choe, et al., 1999a).

dwf5 plants also display short inflorescences and pedicels which are easily converted to wild-type length by exogenous application of Brassinosteroids (FIG. 2). Biochemical complementation of dwf5 mutants with BL as well as 22-OHCR leads to two important conclusions: 1) dwf5 is deficient in endogenous Brassinosteroids, and 2) the biosynthetic defect in dwf5 lies prior to CR. In agreement with the feeding tests, the decreased levels of 24-methylenecholesterol (24-MC) and downstream intermediates, including BL, in dwf5 indicates that the biosynthetic block is located prior to 24-MC and results in a failure to produce bioactive Brassinosteroids. Tracing the metabolites of a ¹³C-labeled precursor has aided in identifying a single step defective in the BR biosynthetic pathway. As explained in the examples, ¹³C-labeled mevalonic acid (MVA) was converted to ¹³C₅-dehydrocampesterol and ¹³C₅-dehydrocampestanol bypassing the Δ⁷ reduction step in dwf5 mutants (FIG. 3). The novel pathway leading to these compounds (FIG. 3) also indicates that the Δ²⁴ and Δ⁵ reduction operates independently of the Δ⁷ reduction step.

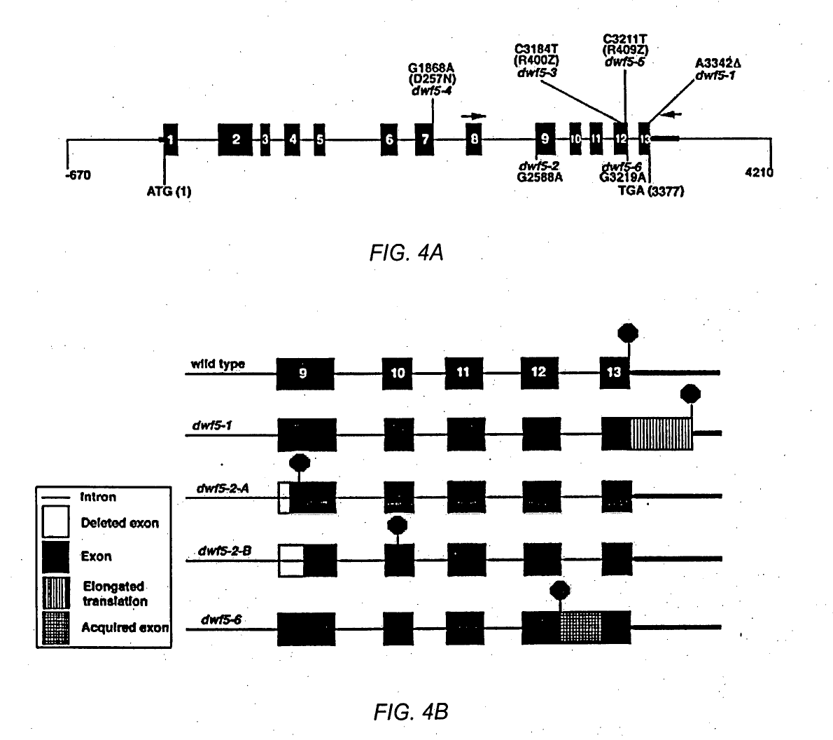

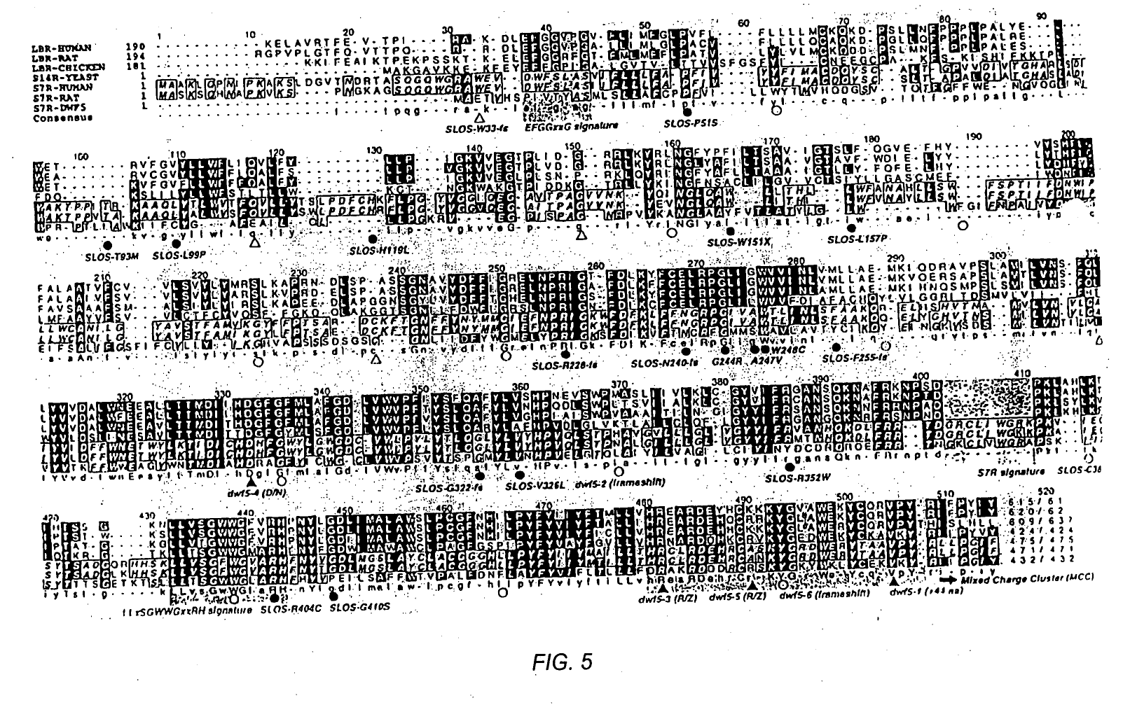

The present invention also pertains to at least six allelic variants of dwf5 (see, FIGS. 4A, 4B and 7). Mutants dwf5-3 and dwf5-5 include premature stop codons in the mixed charge cluster (MCC) domain, resulting in strong mutations. Allelic variant dwf5-4 contains a mutation changing a conserved Asp (D) to Asn (N) located at the starting region of the 6th transmembrane domain (FIG. 5; Fitzky, et al., 1998). The mutation in dwf5-2 abolishes a 3′ splice site, resulting in selection of two cryptic 3′ splice sites downstream of the original one (FIGS. 4A and 4B). Two novel splicing patterns suggest that the 3′ splice site in intron 8 could be selected by either of two different mechanisms commonly found in vertebrates (Simpson and Filipowicz, 1996), namely a scanning process (dwf5-2-A) or by competition of 3′ splice sites based on immediate sequence context (dwf5-2-B). In both types of splicing, premature stop codons are created due to a deletion and a frame shift in exon 9. Therefore, dwf5-2 is likely a null allele because the premature stop codon deletes the C-terminal half of the protein including the 7-reductase signature and the MCC domain. Mutant dwf5-2 possesses the most severe phenotype among the dwf5 alleles. Another splice site mutation (5′-GT to 5′-AT) is found at the 5′ intron splice site of the last intron in dwf5-6. The stop codon created by the unspliced intron deletes half of the MCC domain, suggesting that it is required for normal function of the enzyme.

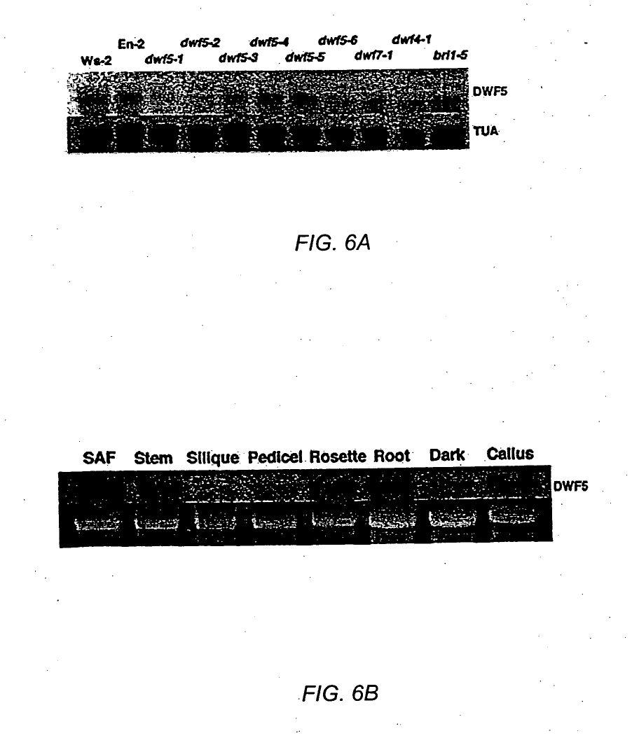

A mutation that appears to affect mRNA stability is found in the dwf5-1 allele. The single base deletion near the C-terminal end is predicted to result in aberrant translation of 54 amino acids before reaching the new stop codon. The newly added amino acid residues form an additional transmembrane domain adjacent to the MCC domain. The steady state mRNA level in dwf5-1 is dramatically lower in the mutant as compared to that of wild type (FIGS. 6A and 6B). Thus, mRNA stability is the likely cause of the mutant phenotype in dwf5-1.

The molecules of the present invention are therefore useful in the production of transgenic plants which display at least one dwf5 phenotype, so that the resulting plants have altered structure or morphology. The present invention particularly provides for altered structure or morphology such as increased or reduced cell length, extended or reduced flowering periods, increased or reduced size of leaves or fruit, increased or reduced branching, increased or reduced seed production and altered sterol composition relative to wild-type plants. The DWF5 polypeptides can be expressed to engineer a plant with desirable properties. The engineering is accomplished by transforming plants with nucleic acid constructs described herein which may also comprise promoters and secretion signal peptides. The transformed plants or their progenies are screened for plants that express the desired polypeptide.

Engineered plants exhibiting the desired altered structure or morphology can be used in plant breeding or directly in agricultural production or industrial applications. Plants having the altered polypeptide can be crossed with other altered plants engineered with alterations in other growth modulation enzymes, proteins or polypeptides to produce lines with even further enhanced altered structural morphology characteristics compared to the parents or progenitor plants.

Isolation of Nucleic Acid Sequences from Plants

The isolation of dwf5 sequences from the polynucleotides of the invention may be accomplished by a number of techniques. For instance, oligonucleotide probes based on the sequences disclosed here can be used to identify the desired gene in a cDNA or genomic DNA library from a desired plant species. To construct genomic libraries, large segments of genomic DNA are generated by random fragmentation, e.g. using restriction endonucleases, and are ligated with vector DNA to form concatemers that can be packaged into the appropriate vector. To prepare a library of tissue-specific cDNAs, mRNA is isolated from tissues and a cDNA library which contains the gene transcripts is prepared from the mRNA.

The cDNA or genomic library can then be screened using a probe based upon the sequence of a cloned gene such as the polynucleotides disclosed here. Probes may be used to hybridize with genomic DNA or cDNA sequences to isolate homologous genes in the same or different plant species. Alternatively, the nucleic acids of interest can be amplified from nucleic acid samples using amplification techniques. For instance, polymerase chain reaction (PCR) technology to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying dwf5-specific genes from plant tissues are generated from comparisons of the sequences provided herein. For a general overview of PCR see Innis et al. eds, PCT Protocols: A Guide to Methods and Applications, Academic Press, San Diego (1990). Appropriate primers for this invention include, for instance, those primers described in the Examples, as well as other primers derived from the dwf5 sequences disclosed herein. Suitable amplification conditions may be readily determined by one of skill in the art in view of the teachings herein, for example, including reaction components and amplification conditions as follows: 10 mM Tris-HCl, pH 8.3, 50 mM potassium chloride, 1.5 mM magnesium chloride, 0.001% gelatin, 200 μM dATP, 200 μM dCTP, 200 μM dGTP, 200 μM dTTP, 0.4 μM primers, and 100 units per mL Taq polymerase; 96° C. for 3 min., 30 cycles of 96° C. for 45 seconds, 50° C. for 60 seconds, 72° C. for 60 seconds, followed by 72° C. for 5 min.

Polynucleotides may also be synthesized by well-known techniques as described in the technical literature. See, e.g., Carruthers, et al. (1982) Cold Spring Harbor Symp. Quant. Biol. 47:411-418, and Adams, et al. (1983) J. Am. Chem. Soc. 105:661. Double stranded DNA fragments may then be obtained either by synthesizing the complementary strand and annealing the strands together under appropriate conditions, or by adding the complementary strand using DNA polymerase with an appropriate primer sequence.

The polynucleotides of the present invention may also be used to isolate or create other mutant cell gene alleles. Mutagenesis consists primarily of site-directed mutagenesis followed by phenotypic testing of the altered gene product. Some of the more commonly employed site-directed mutagenesis protocols take advantage of vectors that can provide single stranded as well as double stranded DNA, as needed. Generally, the mutagenesis protocol with such vectors is as follows. A mutagenic primer, i.e., a primer complementary to the sequence to be changed, but consisting of one or a small number of altered, added, or deleted bases, is synthesized. The primer is extended in vitro by a DNA polymerase and, after some additional manipulations, the now double-stranded DNA is transfected into bacterial cells. Next, by a variety of methods, the desired mutated DNA is identified, and the desired protein is purified from clones containing the mutated sequence. For longer sequences, additional cloning steps are often required because long inserts (longer than 2 kilobases) are unstable in those vectors. Protocols are known to one skilled in the art and kits for site-directed mutagenesis are widely available from biotechnology supply companies, for example from Amersham Life Science, Inc. (Arlington Heights, Ill.) and Stratagene Cloning Systems (La Jolla, Calif.).

Control Elements

Regulatory regions can be isolated from the dwf5 gene and used in recombinant constructs for modulating the expression of the dwf5 gene or a heterologous gene in vitro and/or in vivo. As shown in FIG. 7, the coding region of the dwf5 gene begins at nucleotide position 671 (position 38 of FIG. 8). The region of the gene spanning nucleotide positions 1-633 of FIG. 7 includes the dwf5 promoter. This region may be used in its entirety or fragments of the region may be isolated which provide the ability to direct expression of a coding sequence linked thereto.

Thus, promoters can be identified by analyzing the 5′ sequences of a genomic clone corresponding to the dwf5-specific genes described herein. Sequences characteristic of promoter sequences can be used to identify the promoter. Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. (See, J. Messing et al., in Genetic Engineering in Plants, pp. 221-227 (Kosage, Meredith and Hollaender, eds. (1983)). Methods for identifying and characterizing promoter regions in plant genomic DNA are described, for example, in Jordano et al. (1989) Plant Cell 1:855-866; Bustos et al (1989) Plant Cell 1:839-854; Green et al. (1988) EMBO J. 7:4035-4044; Meier et al. (1991) Plant Cell 3:309-316; and Zhang et al (1996) Plant Physiology 110:1069-1079).

Additionally, the promoter region may include nucleotide substitutions, insertions or deletions that do not substantially affect the binding of relevant DNA binding proteins and hence the promoter function. It may, at times, be desirable to decrease the binding of relevant DNA binding proteins to “silence” or “down-regulate” a promoter, or conversely to increase the binding of relevant DNA binding proteins to “enhance” or “up-regulate” a promoter. In such instances, the nucleotide sequence of the promoter region may be modified by, e.g., inserting additional nucleotides, changing the identity of relevant nucleotides, including use of chemically-modified bases, or by deleting one or more nucleotides.

Promoter function can be assayed by methods known in the art, preferably by measuring activity of a reporter gene operatively linked to the sequence being tested for promoter function. Examples of reporter genes include those encoding luciferase, green fluorescent protein, GUS, neo, cat and bar.

Polynucleotides comprising untranslated (UTR) sequences and intron/exon junctions are also within the scope of the invention. UTR sequences include introns and 5′ or 3′ untranslated regions (5′ UTRs or 3′ UTRs). A dwf5 5′ UTR is shown at nucleotide positions 634-670 of FIG. 7 and nucleotide positions 1-37 of FIG. 8. The 3′ UTR is shown beginning at nucleotide position 4045 in FIG. 7 and spans nucleotide positions 1332-1531 of FIG. 8. As shown in FIG. 4B and FIG. 7, the dwf5 gene sequence includes several introns and exons. These portions of the dwf5 gene especially UTRs, can have regulatory functions related to, for example, translation rate and mRNA stability. Thus, these portions of the gene can be isolated for use as elements of gene constructs for expression of polynucleotides encoding desired polypeptides.

Introns of genomic DNA segments may also have regulatory functions. Sometimes promoter elements, especially transcription enhancer or suppressor elements, are found within introns. Also, elements related to stability of heteronuclear RNA and efficiency of transport to the cytoplasm for translation can be found in intron elements. Thus, these segments can also find use as elements of expression vectors intended for use to transform plants.

The introns, UTR sequences and intron/exon junctions can vary from the native sequence. Such changes from those sequences preferably will not affect the regulatory activity of the UTRs or intron or intron/exon junction sequences on expression, transcription, or translation. However, in some instances, down-regulation of such activity may be desired to modulate traits or phenotypic or in vitro activity.

Use of Nucleic Acids of the Invention to Inhibit Gene Expression

The isolated sequences prepared as described herein, can be used to prepare expression cassettes useful in a number of techniques. For example, expression cassettes of the invention can be used to suppress (underexpress) endogenous dwf5 gene expression. Inhibiting expression can be useful, for instance, in suppressing the phenotype (e.g., dwarf appearance, the Δ⁷ sterol reductase activity) exhibited by dwf5 plants. Further, the inhibitory polynucleotides of the present invention can also be used in combination with overexpressing constructs described below, for example, using suitable tissue-specific promoters linked to polynucleotides described herein. In this way, the polynucleotides can be used to promote dwf5 phenotypes (e.g., activity) in selected tissue and, at the same time, inhibit dwf5 phenotypes (e.g., activity) in different tissue(s).

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al (1988) Proc. Nat. Acad. Sci. USA 85:8805-8809, and Hiatt et al., U.S. Pat. No. 4,801,340.

The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred. It is to be understood that any integer between the above-recited ranges is intended to be captured herein.

Catalytic RNA molecules or ribozymes can also be used to inhibit expression of dwf5 genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, luceme transient streak virus, velvet tobacco mottle virus, solanum nodiflorum mottle virus and subterranean clover mottle virus. The design and use of target RNA-specific ribozymes is described in Haseloffet al (1988) Nature 334:585-591.

Another method of suppression is sense suppression. Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al (1990) The Plant Cell 2:279-289 and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 50%-65%, but a higher identity might exert a more effective repression of expression of the endogenous sequences. Substantially greater identity of more than about 80% is preferred, though about 95% to absolute identity would be most preferred. It is to be understood that any integer between the above-recited ranges is intended to be captured herein. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants which are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Use of Nucleic Acids of the Invention to Enhance Gene Expression

In addition to inhibiting certain features of a plant, the polynucleotides of the invention can be used to increase certain features such as extending flowering, producing larger leaves or fruit, producing increased branching and increasing seed production. This can be accomplished by the overexpression of dwf5 polynucleotides.

The exogenous dwf5 polynucleotides do not have to code for exact copies of the endogenous DWF5 proteins. Modified DWF5 protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski et al (1991) Meth. Enzymol. 194: 302-318). For example, the chains can vary from the naturally occurring sequence at the primary structure level by amino acid substitutions, additions, deletions, and the like. These modifications can be used in a number of combinations to produce the final modified protein chain.

It will be apparent that the polynucleotides described herein can be used in a variety of combinations. For example, the polynucleotides can be used to produce different phenotypes in the same organism, for instance by using tissue-specific promoters to overexpress a dwf5 polynucleotide in certain tissues (e.g., leaf tissue) while at the same time using tissue-specific promoters to inhibit expression of dwf5 in other tissues. In addition, fusion proteins of the polynucleotides described herein with other known polynucleotides (e.g., polynucleotides encoding products involved in the BR pathway) can be constructed and employed to obtain desired phenotypes.

Any of the dwf5 polynucleotides described herein can also be used in standard diagnostic assays, for example, in assays mRNA levels (see, Sambrook et al, supra); as hybridization probes, e.g., in combination with appropriate means, such as a label, for detecting hybridization (see, Sambrook et al., supra); as primers, e.g., for PCR (see, Sambrook et al., supra); attached to solid phase supports and the like.

Preparation of Recombinant Vectors

To use isolated sequences in the above techniques, recombinant DNA vectors suitable for transformation of plant cells are prepared. Techniques for transforming a wide variety of higher plant species are well known and described further below as well as in the technical and scientific literature. See, for example, Weising et al (1988) Ann. Rev. Genet. 22:421477. A DNA sequence coding for the desired polypeptide, for example a cDNA sequence encoding the full length DWF5 protein, will preferably be combined with transcriptional and translational initiation regulatory sequences which will direct the transcription of the sequence from the gene in the intended tissues of the transgenic plant.

Such regulatory elements include but are not limited to the promoters derived from the genome of plant cells (e.g., heat shock promoters such as soybean hsp 17.5-E or hsp17.3-B (Gurley et al. (1986) Mol. Cell. Biol. 6:559-565); the promoter for the small subunit of RUBISCO (Coruzzi et al. (1984) EMBO J. 3:1671-1680; Broglie et al (1984) Science 224:838-843); the promoter for the chlorophyll a/b binding protein) or from plant viruses viral promoters such as the 35S RNA and 19S RNA promoters of CaMV (Brisson et al. (1984) Nature 310:511-514), or the coat protein promoter of TMV (Takamatsu et al. (1987) EMBO J. 6:307-311), cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, heat shock promoters (e.g., as described above) and the promoters of the yeast alpha-mating factors.

In construction of recombinant expression cassettes of the invention, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the T-DNA mannopine synthetase promoter (e.g., the 1′- or 2′-promoter derived from T-DNA of Agrobacterium tumafaciens), and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers such as tissue- or developmental-specific promoter, such as, but not limited to the cell promoter, the CHS promoter, the PATATIN promoter, etc. The tissue specific E8 promoter from tomato is particularly useful for directing gene expression so that a desired gene product is located in fruits.

Other suitable promoters include those from genes encoding embryonic storage proteins. Examples of environmental conditions that may affect transcription by inducible promoters include anaerobic conditions, elevated temperature, or the presence of light. If proper polypeptide expression is desired, a polyadenylation region at the 3′-end of the coding region should be included. The polyadenylation region can be derived from the natural gene, from a variety of other plant genes, or from T-DNA. In addition, the promoter itself can be derived from the DWF5 genomic sequence, as described above.

The vector comprising the sequences (e.g., promoters or coding regions) from genes of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

Production of Transgenic Plants

DNA constructs of the invention may be introduced into the genome of the desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet 16:357-384; Rogers et al (1986) Methods Enzymol. 118:627-641). The Agrobacterium transformation system may also be used to transform, as well as transfer, DNA to monocotyledonous plants and plant cells. (see Hemalsteen et al (1984) EMBO J. 3:3039-3041; Hooykass-Van Slogteren et al (1984) Nature 311:763-764; Griimsley et al (1987) Nature 325:1677-179; Boulton et al (1989) Plant Mol. Biol. 12:31-40.; and Gould et al (1991) Plant Physiol. 95:426-434).

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J. 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

The nucleic acids of the invention can be used to confer desired traits on essentially any plant. A wide variety of plants and plant cell systems may be engineered for the desired physiological and agronomic characteristics described herein using the nucleic acid constructs of the present invention and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the invention has use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea.

One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs of the present invention. Such selection and screening methodologies are well known to those skilled in the art.

Physical and biochemical methods also may be used to identify plant or plant cell transformants containing the gene constructs of the present invention. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods of this invention can be observed by, for example, northern blots of the RNA (e.g., mRNA) isolated from the tissues of interest. Typically, if the amount of mRNA has increased, it can be assumed that the endogenous dwf5 gene is being expressed at a greater rate than before. Other methods of measuring DWF5 activity can be used. For example, cell length can be measured at specific times. Because dwf5 affects the BR biosynthetic pathway, an assay that measures the amount of BL can also be used. Such assays are known in the art. Different types of enzymatic assays can be used, depending on the substrate used and the method of detecting the increase or decrease of a reaction product or by-product. In addition, the levels of DWF5 protein expressed can be measured immunochemically, i.e., ELISA, RIA, EIA and other antibody based assays well known to those of skill in the art, by electrophoretic detection assays (either with staining or western blotting), and sterol (BL) detection assays.

The transgene may be selectively expressed in some tissues of the plant or at some developmental stages, or the transgene may be expressed in substantially all plant tissues, substantially along its entire life cycle. However, any combinatorial expression mode is also applicable.

The present invention also encompasses seeds of the transgenic plants described above wherein the seed has the transgene or gene construct. The present invention further encompasses the progeny, clones, cell lines or cells of the transgenic plants described above wherein said progeny, clone, cell line or cell has the transgene or gene construct.

Polypeptides

The present invention also includes DWF5 polypeptides, including such polypeptides as a fusion, or chimeric protein product (comprising the protein, fragment, analog, mutant or derivative joined via a peptide bond to a heterologous protein sequence (of a different protein)). Such a chimeric product can be made by ligating the appropriate nucleic acid sequences encoding the desired amino acid sequences to each other by methods known in the art, in the proper coding frame, and expressing the chimeric product by methods commonly known in the art.

As noted above, a dwf5 phenotype as defined herein includes any macroscopic, microscopic or biochemical change which is characteristic of over- or under-expression of dwf5. Thus, a DWF5 polypeptide phenotype (e.g., activities) can include any activity that is exhibited by the native DWF5 polypeptide including, for example, in vitro, in vivo, biological, enzymatic, immunological, substrate binding activities, etc. Non-limiting examples of DWF5 activities are described above.

A DWF5 analog, whether a derivative, fragment or fusion of a native DWF5 polypeptide, is capable of at least one DWF5 activity. Preferably, the analogs exhibit at least 60% of the activity of the native protein, more preferably at least 70% and even more preferably at least 80%, 85%, 90% or 95% of at least one activity of the native protein.

Further, such analogs exhibit some sequence identity to the native DWF5 polypeptide sequence. Preferably, the variants will exhibit at least 35%, more preferably at least 50-60%, even more preferably 75% or 80% sequence identity, even more preferably 85% sequence identity, even more preferably, at least 90% sequence identity; more preferably at least 95%, 96%, 97%, 98% or 99% sequence identity.

DWF5 analogs can include derivatives with increased or decreased activities as compared to the native DWF5 polypeptides. Such derivatives can include changes within the domains, motifs and/or consensus regions of the native DWF5 polypeptide, which are described in detail in the examples.

Once class of analogs is those polypeptide sequences that differ from the native DWF5 polypeptide by changes, insertions, deletions, or substitution; at positions flanking the domain and/or conserved residues. For example, an analog can comprise (1) the domains of a DWF5 polypeptide and/or (2) residues conserved between the DWF5 polypeptide, for example as shown in FIG. 5 and described in the examples.

Another class of analogs includes those that comprise a DWF5 polypeptide sequence that differs from the native sequence in the domain of interest or conserved residues by a conservative substitution. For example, an analog that exhibits increased sterol binding can have optimized sterol binding domain sequences that differ from the native sequence.

Yet another class of analogs includes those that lack one of the in vitro activities or structural features of the native DWF5 polypeptides, for example, dominant negative mutants or analogs that comprise a characteristic DWF5 domain but contain an inactivated steroid binding domain.

DWF5 polypeptide fragments can comprise sequences from the native or analog sequences, for example fragments comprising one or more of the domains or regions shown in FIG. 5 and described in the examples.

Fusion polypeptides comprising DWF5 polypeptides (e.g., native, analogs, or fragments thereof) can also be constructed. Non-limiting examples of other polypeptides that can be used in fusion proteins include chimeras of DWF5 polypeptides and fragments thereof; and related polypeptides or fragments thereof, such as those shown in FIG. 5.

DWF5 polypeptides, derivatives (including fragments and chimeric proteins), mutants and analogues may be recombinantly produced, as described above, or can be chemically synthesized. See, e.g., Clark-Lewis et al. (1991) Biochem. 30:3128-3135 and Merrifield (1963) J. Amer. Chem. Soc. 85:2149-2156. For example, DWF5, derivatives, mutants and analogs can be synthesized by solid phase techniques, cleaved from the resin, and purified by preparative high performance liquid chromatography (e.g., see Creighton, 1983, Proteins, Structures and Molecular Principles, W. H. Freeman and Co., N.Y., pp. 50-60). DWF5, derivatives and analog that are proteins can also be synthesized by use of a peptide synthesizer. The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

Further, the dwf5 polynucleotides and DWF5 polypeptides described herein can be used to generate antibodies that specifically recognize and bind to the protein products of the dwf5 polynucleotides. (See, Harlow and Lane, eds. (1988) “Antibodies: A Laboratory Manual”). The DWF5 polypeptides and antibodies thereto can also be used in standard diagnostic assays, for example, radioimmunoassays, ELISA (enzyme linked immunoradiometric assays), “sandwich” immunoassays, immunoradiometric assays, in situ immunoassay, western blot analysis, immunoprecipitationassays, immunofluorescent assays and PAGE-SDS.

Applications

The present invention finds use in various applications, for example, including but not limited to those listed above.

The polynucleotide sequences may additionally be used to isolate mutant dwf5 gene alleles. Such mutant alleles may be isolated from plant species either known or proposed to have a genotype which contributes to altered plant morphology. Additionally, such plant dwf5 gene sequences can be used to detect plant dwf5 gene regulatory (e.g., promoter or promotor/enhancer) defects which can affect plant growth.

The molecules of the present invention can be used to provide plants with increased seed and/fruit production, extended flowering periods and increased branching. The molecules described herein can be used to alter the sterol composition of a plant, thereby increasing or reducing cholesterol content in the plant. A still further utility of the molecules of the present invention is to provide a tool for studying the biosynthesis of brassinosteriods, both in vitro and in vivo.

The dwf5 gene of the invention also has utility as a transgene encoding a Δ⁷ sterol reductase protein that mediates one or more steps in brassinosteriod biosynthesis which results in a transgenic plant to alter plant structure or morphology. The dwf5 gene also has utility for encoding the DWF5 protein in recombinant vectors which may be inserted into host cells to express the DWF5 protein. Further, the dwf5 polynucleotides of the invention may be utilized (1) as nucleic acid probes to screen nucleic acid libraries to identify other enzymatic genes or mutants; (2) as nucleic acid sequences to be mutated or modified to produce DWF5 protein variants or derivatives; (3) as nucleic acids encoding the sterol Δ⁷ reductases in molecular biology techniques or industrial applications commonly known to those skilled in the art.

The dwf5 nucleic acid molecules may be used to design antisense molecules, useful, for example, in gene regulation or as antisense primers in amplification reactions of dwf5 gene nucleic acid sequences. With respect to dwf5 gene regulation, such techniques can be used to regulate, for example, plant growth, development or gene expression. Further, such sequences may be used as part of ribozyme and/or triple helix sequences, also useful for dwf5 gene regulation.

The dwf5 control element (e.g., promoter) of the present invention may be utilized as a plant promoter to express any protein, polypeptide or peptide of interest in a transgenic plant. In particular, the dwf5 promoter may be used to express a protein involved in brassinosteriod biosynthesis.

The DWF5 protein of the invention can be used in any biochemical applications (experimental or industrial) where sterol Δ⁷ reductase activity is desired, for example, but not limited to, regulation of BL synthesis, regulation of other sterol synthesis, modification of elongating plant structures, and experimental or industrial biochemical applications known to those skilled in the art.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Restriction and modifying enzymes, as well as PCR reagents were purchased from commercial sources, and used according to the manufacturers' directions. In the cloning of DNA fragments, except where noted, all DNA manipulations were done according to standard procedures. See, e.g., Sambrook et al., supra. Restriction enzymes, T₄ DNA ligase, E. coli, DNA polymerase I, Klenow fragment, and other biological reagents were purchased from commercial suppliers and used according to the manufacturers' directions.

### EXAMPLE 1

### Isolation of Six Independent dwf5 Alleles

Fourteen dwarf mutants resembling dwf1 (Feldmann, et al., 1989) were isolated from mutant populations induced by T-DNA insertion mutagenesis. dwf5-1 was identified among these mutants. When dwf5-1 was crossed to the other dwf loci, dwf1, bri1/dwf2, cpd/dwf3, dwf4, det2/dwf6, and dwf7/ste1-2, the F1 plants were wild type, showing that dwf5 belonged to a novel locus.

Although dwf5-1 was found in a population of T-DNA generated transformants, the mutant failed to cosegregate with the kanamycin-resistance marker, suggesting that dwf5-1 is an untagged mutant. Two additional alleles, in the Enkheim-2 ecotype, were identified among the dwarf mutants obtained from the Nottingham Arabidopsis Stock Center (NASC, University of Nottingham, UK). These alleles include dwf5-2 (NASC stock number N398) and dwf5-3 (N402). In addition, screening of EMS-induced mutant populations (Wassilewskija-2 background) resulted in two alleles, dwf5-5 and dwf5-6.

dwf5 was mapped using simple sequence length polymorphisms (SSLP) markers (Bell and Ecker, 1994). The meiotic recombination ratio between dwf5 and three SSLP markers on chromosome 1 was 18/68 with nga111, 6/98 with nga280, and 23/68 with nga392. These data placed dwf5 6 cM south of nga280. Since a mutant called lepida (le) was similar to dwf5 in morphology (McKelvie, 1962) and map position (Kranz and Scheidemann, 1978), we also made crosses between dwf5-1 and le. The offspring of this cross also displayed a dwarf phenotype, indicating that le (dwf5-4) and dwf5 were allelic.

All six dwf5 alleles segregated as recessive mutants.

### EXAMPLE 2

### Morphological Analysis of dwf5

Standard procedures for plant growth, measurements on floral organs, scoring the number of seeds in a silique, biochemical complementation tests with Brassinosteroids, analysis of endogenous Brassinosteroids, and 13C-labeled mevalonic acid feeding experiments have been described previously (Choe, et al., 1999b).

Table 1 presents the results of a morphometric analysis of wild type, dwf5 alleles, and dwf4-1. The morphometric analysis in Table 1 was performed 6 weeks after germination. The height of more than 15 individual plants was measured to the nearest centimeter. Seed size was measured using photographically enlarged pictures of wild-type and dwf5-1 seeds. The length and width of 30 different seeds of the Wassilewskija-2 (Ws-2) wild type and dwf5-1 were measured. Numbers in Table 1 represent averages with associated standard deviations.

TABLE 1

| | Height at | | | | | Number |
|---|---|---|---|---|---|---|
| | 6 weeks | Flower (mm)² | | Fruit (mm)³ | | of seeds |
| | (cm)¹ | Gynoecium | Stamen | Pedicel | Silique | in a pod⁴ |
| Ws-2 | 34.3 ± 3.2 | 2.35 ± 0.02 | 2.51 ± 0.02 | 12.6 ± 1.7 | 13.7 ± 2.0 | 49.7 ± 5.1 |
| dwf5-1 | 9.2 ± 1.0 | 1.95 ± 0.03 | 2.06 ± 0.02 | 7.9 ± 1.2 | 8.1 ± 0.5 | 46.3 ± 5.1 |
| dwf5-2 | 7.6 ± 1.3 | 2.09 ± 0.02 | 1.78 ± 0.01 | 4.4 ± 0.7 | 5.1 ± 0.7 | 13.0 ± 3.2 |
| dwf5-3 | 8.5 ± 1.8 | 2.02 ± 0.05 | 1.83 ± 0.06 | 7.0 ± 1.8 | 6.7 ± 1.6 | 11.3 ± 1.8 |
| dwf5-4 | 11.1 ± 0.8 | 2.35 ± 0.02 | 2.06 ± 0.06 | 6.4 ± 0.9 | 8.6 ± 0.6 | n.a.⁵ |
| dwf5-5 | 8.3 ± 1.7 | n.a. | n.a. | 6.9 ± 1.1 | 5.8 ± 0.8 | n.a. |
| dwf5-6 | 18.1 ± 0.9 | n.a. | n.a. | 6.3 ± 0.9 | 7.0 ± 0.5 | n.a. |
| dwf4-1 | 4.2 ± 0.7 | 1.61 ± 0.04 | 0.99 ± 0.04 | 2.6 ± 0.4 | 3.4 ± 0.6 | 0.0 ± 0.0 |

¹measured to the nearest cm (n = 15)
²measured to the nearest 1/10 mm (n = 5)
³measured to the nearest cm (n = 30)
⁴n = 15
⁵Not analyzed

The dwarf phenotype is typical of Arabidopsis BR dwarfs. The phenotype includes small, dark-green, round leaves, short internodes and siliques, and increased number of inflorescences. dwf5 mutants are larger than dwf4 in height with a concomitant increase in fertility. Among the dwf5 mutants, dwf5-2 and dwf5-3 are the most severe.

Key characteristics of dwf5 mutants include a short robust stature, short internodes, an increased number of inflorescences, and dark-green, round leaves similar to the phenotype previously reported for the other BR dwarfs (Azpiroz, et al., 1998; Choe, et al., 1999a, 1999b). The height of dwf5 plants was variable depending on the allele (Table 1), ranging from 22 to 53% of wild-type height at 6 weeks of age. The lengths of the petioles, pedicels, and sliliques were also reduced in dwf5 alleles.

Silique length was correlated to fertility. The longer siliques of dwf5-1 contained more seeds relative to the shorter ones of dwf5-2 and dwf5-3 (Table 1). The number of seeds in dwf5-1 siliques was the highest among the dwf mutants reported to date (Table 1; Azpiroz, et al., 1998; Choe, et al., 1999a; Choe, et al., 1999b). In addition, dwf5-1 is the only BR mutant that has been shown to possess stamens that are longer than the gynoecium similar to wild type (Table 1). In all other dwf5 alleles as well as other BR dwarf mutants, the stamens are shorter than the gynoecium.

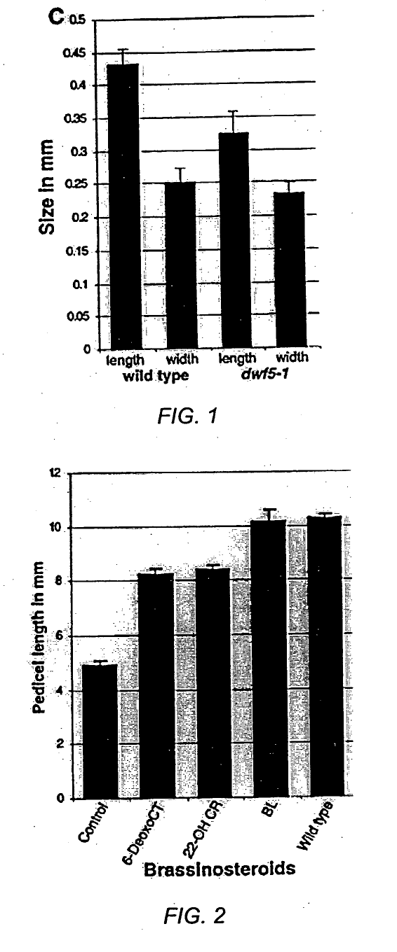

In spite of the increased fertility in dwf5 mutants, dwf5 seeds did not develop normally. Wild-type seeds have a light-brown color and oval shape while dwf5-1 seeds have a dark-brown color and round shape. Relative to the light-brown, oval shape of the wild type, dwf5-1 seeds were darker-brown with a wrinkled morphology. The length and width of wild-type and dwf5-1 seeds is given in FIG. 1 (n=30). The average size in mm with standard deviation is shown. (Bar=1 mm).

dwf5 seeds did not germinate well compared to wild type; >90% of wild-type seeds germinated on agar-solidified Murashige and Skoog media, whereas <60% of dwf5-1 seeds germinated. This reduced germination was partially corrected by supplementing the germination media with 10⁻⁷ M brassinolide. The reduced germination of dwf5 seeds may be a secondary effect of retarded growth, or possibly DWF5 is involved in seed development and/or germination.

### EXAMPLE 3

### Biochemical Characterization of dwf5

It was previously shown that dwf7 displays reduced cell elongation, and that the short cells could be reverted to wild-type length by exogenous application of Brassinosteroids (Choe, et al., 1999b). The ability of Brassinosteroids to rescue the phenotype of dwf5 was tested. The data presented in FIG. 2 show the biochemical complementation of dwf5-1 plants with brassinolide (BL) and its biosynthetic intermediates. In the figure, the results of application of 6-deoxocathasterone (6-DeoxoCT), 22-hydroxycampesterol (22-OHCR), and brassinolide (BL) are significant increases in dwf5 pedicel length compared to a water-treated control. The length of pedicels was measured 7 days after application (n=15). Data points represent the average with one standard deviation. Further, the BR biosynthetic intermediate 6-deoxocathasterone (6-DeoxoCT), and the synthetic compound 22-hydroxycampesterol (22-OHCR) induced pedicel growth up to 80% of wild-type length.

In addition, BL application completely rescued the length of dwf5 pedicels to that of wild type, suggesting that dwf5 is deficient in endogenous brassinosteroids. Biochemical complementation by 6-deoxoCT and 22-OHCR also indicated that the defective biosynthetic step is prior to campesterol (CR). 22-OHCR has been used in feeding tests because the biosynthetic intermediates prior to campestanol (CN) were found to have undetectable activity in bioassays (Choe, et al., 1999a; Choe, et al., 1999b; Ephritikhine, et al., 1999).

Because the biochemical feeding tests suggested that dwf5 was defective in one of the steps prior to CR, the endogenous levels of CR and downstream compounds were evaluated using GC-selective ion monitoring (GC-SIM). The levels of BL and its biosynthetic intermediates were examined both in wild type and dwf5.

Scarcity of the sterols and BR biosynthetic intermediates further confirmed that the defective biosynthetic reaction in dwf5 is prior to 24-methylenecholesterol (24-MC). To pinpoint the single step disrupted in dwf5, metabolism tests were performed with ¹³C-labeled mevalonic acid (MVA). FIG. 3 shows the results of biochemical analyses of the metabolism of ¹³C-MVA. Important carbon atoms are numbered at the episterol structure. ¹³C-labeled MVA and compactin, an endogenous MVA biosynthetic inhibitor, were fed to wild-type and dwf5 seedlings. MVA was successfully converted through the sterol biosynthetic pathway in wild type, whereas conversion is blocked at the sterol Δ⁷ reduction step in dwf5. In FIG. 3, solid lines represent a single step, whereas broken lines indicate more than one step (Unit=μg/5 g fresh weight).

The basic methodologies for this experiment were described previously (Choe, et al., 1999b). As shown in FIG. 3, ¹³C-MVA was converted to ¹³C₅-episterol and subsequent sterols such as ¹³C₅-24-MC and ¹³C₅-CR in the Ws-2 wild type, but not in dwf5. ¹³C₅-5-dehydroepisterol was not detected in either wild type or dwf5 possibly due to rapid conversion to the downstream compounds. Interestingly, a novel bypass pathway was created in dwf5. Unlike wild type, ¹³C-MVA was converted to ¹³C₅-7-dehydrocampesterol and 13C₅-7-dehydrocampestanol. Two lines of evidence, a failure to detect ¹³C₅-24-MC and ¹³C₅-CR in dwf, and conversion of ¹³C-MVA to ¹³C₅-7-dehydrocampesterol and ¹³C₅-7-dehydrocampestanol possibly via ^{—}C₅-5-dehydroepisterol, suggest that the biosynthetic steps up to 5-dehydroepisterol are active, whereas the Δ⁷ reduction step is missing in the dwf5 mutants.

The results suggest that a novel pathway leading to 7-dehydrocampestanol is operating in dwf5 plants. Further details of the known components of this pathway can be found in the reference of Choe, et al., 1999b.

Experiments performed in support of the present invention indicated the following. The endogenous levels of 24-MC, CR, and CN are scarce in both weak (dwf5-1) and severe (dwf5-2) alleles. The relative ratio of 24-MC, CR, and CN to that of wild type was 2.4, 0.4, 3.7%, and 0.5, 0.2, 1% in dwf5-1 and dwf5-2, respectively. The difference in the endogenous levels of sterols in these two alleles is reflected in the degree of severity in that dwf5-2 is shorter and less fertile than dwf5-1. As a consequence of reduced sterol levels, Brassinosteroids were not detected in this analysis.

In addition to their role as precursors of Brassinosteroids, plant sterols play an important role in membranes as structural components. Membrane sterols affect the function of membrane-associated proteins by changing membrane fluidity (Bach and Benveniste, 1997). A phenotypic comparison of Arabidopsis dwarf mutants in the sterol pathway (dwf1, dwf5, and dwf7/ste1) versus those in the BR-specific pathway (det2, cpd, and dwf4) has not resulted in any obvious differences that might be attributable to a deficiency in membrane sterols. It is possible that the role of sterols e.g., sitosterol, stigmasterol, campesterol, was substituted by novel sterols, such as 7-dehydrocampesterol, or by modulating the fatty acid content in membranes. More precise analysis of dwf5 membranes e.g., ion leakage tests after heat or cold treatment, may show differences between dwf5 and wild-type membranes specifically caused by the sterol deficiency.

### EXAMPLE 4

### Molecular Cloning and Characterization of dwf5

Experiments performed in support of the present invention (described above) indicated that the biosynthetic defect in dwf5 lies at the S7R reaction. In view of these findings, DNA sequence databases (e.g., GenBank, National Center for Biotechnology Information, Bethesda Md.; http://www.ncbi.nlm.nih.gov) were searched for candidate genes whose products were associated with the S7R reaction. An Arabidopsis S7R cDNA sequence was found in GenBank (Lecain, et al., 1996). To test if the S7R gene location corresponded to dwf5 the two markers were genetically mapped relative to each other. A cleaved amplified polymorphic sequence (CAPS) marker that was developed with the S7R gene cosegregated with dwf5, indicating linkage (0 out of 40 chromosomes).

To determine the map position of a candidate S7R gene (GenBank accession number U49398), a CAPS marker (Konieczny and Ausubel, 1993) for the locus was created and the linkage of this polymorphism to dwf5 was tested. Routine molecular techniques for DNA and RNA handling were performed according to Sambrook, et al. (1989). Some of the oligonucleotide sequences employed were as follows (sequences are given in the 5′ to 3′ direction):

| | | |
|---|---|---|
| DW5_FF, | GTGTGAGTAATTTAGGTCAACACAGATCA; | (SEQ ID NO:_) |
| DW5_LR, | GGCTCGGTCTTTTGATGATTCCAACGTT; | (SEQ ID NO:_) |
| DW5_2F, | TGTGGTAACCTAATAATTGACTTCTATT; | (SEQ ID NO:_) |
| DW5_2R, | GGAGAAGTGTAGACAGAAGGCACCCACACT; | (SEQ ID NO:_) |
| DW5_3F, | ATTGGAACACCATGGACATTGCACATGAC; | (SEQ ID NO:_) |
| DW5_4F, | AGGGTCCAATATCTCCAGCCGGAAACCGA; | (SEQ ID NO:_) |
| DW5_4R, | GAAAATATTTCACCCAAGTGATCATAGA; | (SEQ ID NO:_) |
| DW5_5F, | GGGTGCCTTCTGTCTACACTTCTCCAG; and | (SEQ ID NO:_) |
| DW5_5R, | AAATGACGAGCCAATCCCCA. | (SEQ ID NO:_) |

In the primer designations the underlined space was used to distinguish forward or reverse primers from the gene acronym DW5, e.g., DW5_{—}5R. Because only a cDNA sequence was available for S7R, possible exon-intron junctions were predicted before designing primers using the RNASPL utility available at the Baylor College of Medicine (Houston, Tex.; http://www.hgsc.bcm.tmc.edu/SearchLauncher/). After sequences were determined the primers were purchased from Sigma-Genosys Biotechnologies, Inc. (The Woodlands, Tex.). Template DNA for PCR was isolated from 2 or 3 leaves (Krysan, et al., 1996). The longest PCR product spanning the whole coding region was found to be 3.5 kb in the amplification using DW5_FF and DW5_LR primers. Of the primer sets, DW5_{—}3F and DW5_LR yielded a 1.6 kb DNA fragment corresponding to the 3′ half of the gene. This PCR product was further analyzed to detect polymorphisms among ecotypes. One-tenth of the PCR amplification products from each ecotype was subjected to a restriction digestion with enzymes that were predicted to restrict the cDNA sequence of Columbia ecotype (GenBank Accession No. U49398). HaeIII cuts Columbia DNA once to produce 0.9 kb and 0.7 kb fragments, whereas Ws-2 and Ler DNA were unrestricted. This polymorphism was used to test for cosegregation with dwf5.

To make a mapping population, F1 seeds were obtained by crossing dwf5-1 to Columbia wild type. Fifty different dwarf plants from the F2 generation were selected for genomic DNA isolation. Individual PCR products were amplified using DW5_{—}3F and DW5 LR primers and the amplification products were digested with HaeIII. These restriction digestion samples were run on 1% TAE (40 mM Tris-acetate and 10 mM EDTA) agarose gel to resolve the digestion fragments and determine the restriction patterns. The mapping results indicated that the S7R gene was linked to dwf5.

DNA samples corresponding to dwf5 alleles were sequenced at the Arizona Research Laboratory (Tucson, Ariz.) using an ABI-377 automated sequencer (Applied Biosystems, Foster City, Calif.). Putative mutations were identified by comparing the sequence of mutant DNA with that of the wild-type background from which the mutant alleles were derived. The putative mutation was confirmed by repeated sequencing (>2) of independent PCR products for each strand. Sequence analysis was performed using software packages, such as Genetics Computer Group (Madison, Wis.), DNASTAR (Madison, Wis.), and utilities available at the Arabidopsis web site (http://genome-www.stanford.edu/Arabidopsis/). A summary of the genomic sequences of DWF5 and dwf5 mutants are presented in FIG. 7. FIG. 8 shows a sequence of a DWF5 cDNA and the corresponding translation product. Further, a summary of the six independent alleles of Arabidopsis dwf5 and their corresponding mutations are presented in Table 2.

TABLE 2
Six independent alleles of Arabidopsis dwf5 and their mutations.

| | Previous | | | Mutations in sequence | |
|---|---|---|---|---|---|
| Allele | name | Ecotype | Mutagen | Genomic DNA (4880 bp) | Amino acid (432 aa) |
| dwf5-1 | 2 | Ws-2¹ | T-DNA² | single bp deletion (3342³) | elongated C-terminus (+44 aa) |
| dwf5-2 | N398⁴ | En-2⁵ | unknown | G2588⁵A, 3′ splice site of | stops at 298, 344, or more |
| | | | | intron 8 | |
| dwf5-3 | N402⁴ | En-2 | unknown | C3184⁵T | Arg to stop (400) |
| dwf5-4 | lepida | Estland | unknown | G1868⁵A | Asp to Asn (257) |
| dwf5-5 | wm9-4 | Ws-2 | EMS⁶ | C3211⁵T | Arg to stop (409) |
| dwf5-6 | marial | Ws-2 | EMS | G3219⁵A, 5′ splice site of | Arg to stop (411) |
| | | | | intron 12 | |

¹Wassilewskija-2
²transfer DNA of Agrobacterium Ti plasmid
³Position when adenine of the translation initiation codon is set as 1
⁴Nottingham Arabidopsis Stock Center number (NASC, University of Nottingham, UK)
⁵Enkheim-2.
⁶Ethylmethanesulfonate

Alignments of the resulting sequences are schematically presented in FIGS. 4A and 4B. FIGS. 4A and 4B were made using the Gene Construction Kit (Textco, West Lebanon, N.H.) and box shading of the multiple sequence alignment was done using ALSCRIPT (Barton, 1993). The organization of dwf5 was determined by comparing the sequences of genomic DNA with the cDNA. FIG. 4A displays a schematic of the dwf5 gene which consists of 13 exons and 12 introns. All 12 introns were bordered by 5′-GT and AG-3′ splice site sequences. A single mutation was found in the S7R sequence from all six alleles.

The illustration in FIG. 4A shows that dwf5 contains 13 exons (filled rectangles) and 12 introns (line). The 5′(37 bp) and 3′(198 bp) untranslated regions are shown in thicker lines. The position of adenosine in the translation start codon ATG was set at position 1. The six independent mutant alleles are shown with base changes and amino acid changes in parentheses. Arrows correspond to positions of PCR primers used for RT-PCR described below.

The illustration in FIG. 4B shows a schematic view of the region from exon 9 to the 3′ UTR (based on sequencing data). The position of the stop codon is depicted with a hexagonal stop sign. The dwf5-1 mutation extended translation to add 54 aberrant amino acids and to shorten the 3′ UTR to 65 bp. Two major types of aberrant splicing patterns were identified: dwf5-2-A and dwf5-2-B chose cryptic 3′ splice sites 25 bp and 62 bp downstream of the original, respectively. These aberrant splicing patterns caused frameshifts and eventually introduced premature stop codons. In dwf5-6, the mutation was found in the 5′ splice site of intron 12. The major splicing defect in dwf5-6 was a failure to splice out the last in the mRNA, resulting in creation of a premature stop codon.

To confirm that the S7R gene encodes DWF5, the genomic DNA of Ws-2, En-2, and Estland wild types, were sequenced and compared to sequence obtained from the mutant alleles, described herein, of dwf. Genomic DNA flanking the dwf5 cDNA was isolated by thermal asymmetric interlaced PCR (Liu, et al., 1995). The following two sets of nested primers were used to amplify each of the 3′ and 5′ flanking DNAs (the oligonucleotide sequences are presented in the 5′ to 3′ orientation):

| | | |
|---|---|---|
| D5-3-1, | TTACTCTGATTTGCTGACAATATTCGGGTTTTG; | (SEQ ID NO:_) |
| D5-3-2, | GTAAAAAGGTATGGGAAATATTGGAAGCTGTAT; | (SEQ ID NO:_) |
| D5-3-3, | ATTGTAACGAAGTCTGTTGTTCTCATTTTCTAC; | (SEQ ID NO:_) |
| D5-5-1, | AGGAGCCAGAAAAGTGTGCGAGTC; | (SEQ ID NO:_) |
| D5-5-2, | CAGGAGAATGACGAAAGGTGGACA; | (SEQ ID NO:_) and |
| D5-5-3, | TGGACAGAAGGCGAGAAGCGATAA. | (SEQ ID NO:_) |

Arbitrary degenerate primers, AD1, AD2, and AD3 were synthesized following the sequence described by Liu et al. (1995).

The results of these analyses indicated that the S7R gene encodes dwf5.

Based on the sequence comparisions described above (FIGS. 4A and 4B), altered splicing patterns were expected to be found in plants bearing the two alleles, dwf5-2 and dwf5-6. Evaluation of the mRNA products of these alleles was carried out by RT-PCR and subsequent sequencing of the resultant PCR products.

RT-PCR was carried out to synthesize DWF5 cDNA from the wild-type and dwf5 mutants. Total RNA was subject to DNAase I treatment (Boehringer Manheim-Roche, Indianapolis, Ind.) to remove genomic DNA. RNA was purified from the DNAase reaction using a RNEASY MINI KIT (Qiagen, Santa Clarita, Calif.). cDNA was first synthesized using reverse transcriptase (BRL, Gaithersburg, Md.) with a poly T primer called A1T17 (5′-GGCCACGCGTCGACTAGTACTTTTTTTTTTTTTTTTT-3′) (SEQ ID NO:______). dwf5 cDNA spanning the whole coding region was amplified using primers D5WKPN-F (5′ ATCGGTACCAAGCAGAAGAAGAAAATGGCGGAG-3′) (SEQ ID NO:______) and D5BAM-5 (5′-ATCGGATCCGCATTTTTGTTTTGGCTCGGTCTTTTGA-3′)(SEQ ID NO:______).

To amplify the possible misspliced region, A1T17-primed total cDNA was used as a template for PCR with DW5_{—}5F and DW5_LR primers. PCR for wild-type genomic DNA was also performed to check if there was any undesired amplification from residual genomic DNA in the RT-PCR.

One-tenth of each of the PCR amplification products was fractionated on 1.1% agarose gel, and the DNA was transferred to a membrane for DNA gel blot hybridization using dwf5 cDNA as a probe. As size controls, wild-type genomic DNA (WT-G) and cDNA (WT-C) were amplified in conjunction with RT-PCR of dwf5-2 and dwf5-6 cDNA. Two major DNA fragments in the dwf5-2 lane and one band from dwf5-6 were gel-purified using Prep-A-Gene kit (Bio-Rad, Hercules, Calif.) and sequenced to confirm the splice pattern shown in FIG. 4B. These results are further discussed in Example 6 (below).

The PCR product of the wild-type gene DWF5 was cloned into a pCR 2.1-TOPO TA cloning vector (Invitrogen, Carlsbad, Calif.), and sequenced to check for any PCR errors before subcloning into the KpnI and BamHI sites of pART7, which are designed to overexpress the cDNA under the control of the 35S promoter. pART7 is a bridge plasmid for the plant binary vector pART27 system (Gleave, 1992). This overexpression construct was designated AOD5 (Arabidopsis Overexpressor of DWF5).

### EXAMPLE 5

### Sequence Alignment and Identification of Motifs

To examine the degree of identity between sterol reductases, as well as to validate the biological importance of dwf5 mutations in the context of the protein sequences, multiple sequence alignments were performed using the six protein sequences showing the highest similarity with dwf5 in a BLAST search (Altschul, et al., 1997). The sequences included human (Accession no. NM_{—}001360) and rat (AF057368) S7Rs, yeast sterol Δ¹⁴ reductase (JC4057), and lamin B receptors (LBR) isolated from human (NM_{—}002296), rat (AB002466), and chicken (P23913). The overall amino acid identity between dwf5 and other sequences was less than 40%, with many residues being highly conserved between the sequences.

The results of the multiple sequence alignment of dwf5 with similar sequences are presented in FIG. 5. The sequence for DWF5 is shown in FIG. 8 and the sequences of the dwf5 mutants are presented in FIG. 7. GenBank accession numbers for the other sequences are as follows: NM_{—}001360 (S7R-human, human sterol Δ⁷ reductases), AF057368 (S7R-rat), JC4057 (S14R-yeast, yeast sterol C-14 reductase), and NM_{—}002296 (human LBR), AB002466 (rat LBR), and P23913 (chicken LBR).

In FIG. 5, about 200 N-terminal amino acids of all LBRs and 29 C-terminal amino acids of the chicken LBR were truncated to maximize alignment. Amino acid residues conserved more than 50% among the 7 compared sequences are shown in inverse characters. Amino acid residues conserved among sterol Δ⁷ reductases (S7R) are boxed. Positions for the Arabidopsis dwf5 mutations are annotated with filled triangles and described underneath. Mutations causing Smith-Lemli-Opitz syndrome (SLOS) are shown with filled circles according to Fitzky et al. (1998). “fs” refers to frameshift mutations. Introns are indicated with open circles (Arabidopsis) or open triangles (human DHCR7). Previously reported domains, EFGGxxG and LLxSGWWGxxRH, a newly identified S7R signature, and the mixed charge cluster are shaded and labeled. Dashes in protein sequences indicate gaps introduced to maximize alignment. A consensus sequence is shown in the bottom row of the alignment. Dashes in the consensus mean <50% identity among the 7 sequences compared. Capital letters stand for residues conserved among all 7 sequences, whereas lower case letters mean 50% identity. Multiple sequence alignment was performed using PILEUP in the Genetics Computer Group software (Madison, Wis.) with a gap creation penalty of 4 and a gap extension parameter of 1.

As indicated in FIG. 5, the previously reported LLxSGWWGxxRH and EFgGxxG signatures (Lecain, et al., 1996) (upper case for fully identical residues, lower case if conserved >50%, and x for variable residues) were identified at the N-terminus. In addition, it was found that the GrCLiWGrk signature is only conserved in Δ⁷ reductases but missing in other reductases (FIG. 5).

Furthermore, it has been proposed that the last 40 amino acids consist of a hydrophilic soluble domain (Moebius, et al., 1998). Analysis of protein secondary sequence using the Statistical Analysis of Protein Sequence (SAPS) program (Brendel, et al., 1992) showed that this soluble domain encompasses a significant mixed charge cluster (MCC) from 399 to 426. Sixteen of the clustered 28 amino acids had either a positive or negative charge. The unusual clustering of charged amino acid residues suggests that this moiety plays an important role for proper function in this group of enzymes. In support of this, many of dwf5 mutations were directly associated with this domain by deleting part or all of it.

To summarize, the dwf5 sequence was most similar to human and rat S7R5. Yeast sterol Δ¹⁴ and Δ²⁴ reductases, and the C-terminal 400 amino acids of human and chicken LBR also showed significant similarity (FIG. 5). LBRs show identity with dwf5 because the 400 amino acids of the C-terminal domain reportedly possess a Δ¹⁴ reductase activity (Silve, et al., 1998), whereas the remaining N-terminal domain has been proposed to be involved in nuclear assembly during the cell cycle (Gant and Wilson, 1997). The combined function of the whole protein is as yet unclear. Lecain et al. (1996) first identified two types of signature sequences in Δ⁷ reductases. One group of amino acid residues, LLxSGWWGxxRH (SEQ ID NO:______) was commonly conserved in all of the sterol reductases, whereas the other group, EFGGxxG (SEQ ID NO:______), distinguished Δ⁷ reductases from other sterol reductases (FIG. 5). In addition to these, our sequence alignment revealed two additional domains both located in the C-terminal half of the protein. First, a signature consisting of GrCLiWGrk (SEQ ID NO:______) was only found in S7R sequences (FIG. 5), suggesting a specific role in these enzymes. Second, an unusual cluster of charged amino acid residues was identified at the C-terminal end of all the reductase sequences. A mixed charge cluster (MCC) is highly conserved within the cut family of homeodomains proteins, suggesting an important role in this group of proteins (Brendel, et al., 1992). Similarly, it is likely that the terminal MCC domain of the sterol reductases plays a pivotal role for the proper function of the enzymes. In support of this, most of the dwf5 mutations described in the examples below were directly associated with this domain. dwf5-3 and dwf5-5 carried premature stop codons in the MCC domain, resulting in strong mutations. dwf5-4 contained a mutation changing a conserved Asp (D) to Asn (N) located at the starting region of the 6th transmembrane domain (FIG. 5; Fitzky, et al., 1998). In relation to this, many of the mutations in human SLOS patients were found in or near the putative transmembrane domains (Fitzky, et al., 1998).

Most of the sterol reductases that are involved in sterol and BR-specific biosynthesis have been isolated from Arabidopsis. These include reductases for Δ⁷ (DWF5), Δ²⁴ (DWF1), and Δ⁵ (DET2). It has been shown that the human steroid Δ⁵ reductase gene expressed using the 35S promoter in det2 could complement the mutant phenotype, suggesting that the function of Δ⁵ reductases are conserved between humans and Arabidopsis (Li and Chory, 1997). However, in spite of significant sequence identity (FIG. 5), the function of the S7R was marginally conserved between Arabidopsis and humans. Ectopic overexpression of the human S7R gene in dwf5 did not induce significant complementation of the dwf5 phenotype. Furthermore, none of the intron positions were shared between the Arabidopsis and human S7R sequences (FIG. 5). Phylogenetically, in contrast to the Δ⁵ reductase gene, the other plant and animal reductases appear to show independent evolution.

### EXAMPLE 6

### Splice Site Mutations in dwf5-2 and dwf5-6

Two mutants, dwf5-2 and dwf5-6, possessed splice site mutations. dwf5-2 carried a mutation at 2588, numbered relative to the start codon (position 3258 of the sequence shown in FIG. 7), abolishing a 3′ splice site (AG-3′) by changing it to AA-3′ which resulted in aberrant splicing of intron 8. To determine the splicing patterns and their effects on translation, we performed a reverse transcriptase polymerase chain reaction (RT-PCR). Two different DNA fragments derived from aberrant splicing patterns were amplified. Comparison of the cDNA sequences from wild type and dwf5-2 revealed that two different cryptic recognition sites were selected. dwf-2-A used a cryptic site 25 bp downstream of the original one, while dwf5-2-B used one 62 bp downstream. These splicing patterns are summarized in FIG. 4B. The use of the downstream cryptic sites resulted in partial deletion of exon 9, leading to the introduction of premature stop codons. The premature stop codons created in dwf5-2 are the N-terminal-most found in the dwf5 mutants (FIG. 5), and the phenotype of dwf5-2 is the most severe among the dwf5 alleles (Table 1). In addition, lightly hybridizing bands in the dwf5-2 lane (FIG. 4B) indicated that there may be additional types of mis-splicing as minor events.

Another frame shift mutation caused by mis-splicing was found in dwf5-6. A guanosine at 3219, numbered relative to the start codon (position 3889 of the sequence shown in FIG. 7) was changed to adenosine, resulting in removal of the 5′ splice site of the last intron (5′-GT to 5′-AT). As with dwf5-2, RT-PCR analysis was employed to detect mis-spliced mRNA. The size of the transcript in dwf5-6 was larger than that of the wild-type control (WT-C lane), suggesting that the last intron was not spliced out. This was confirmed by sequencing the cDNA made from dwf5-6 transcripts. A failure to splice out the intron is predicted to create a stop codon at position 3219 (position 3889 of the sequence shown in FIG. 7), deleting half of the C-terminal mixed charge cluster (MCC; FIG. 5).

### EXAMPLE 7

### Nonsense and Missense Mutations in dwf5-3, dwf5-4, and dwf5-5

Three mutants contained point mutations, introducing premature stop codons (dwf5-3 and dwf5-5) and a substitution of a conserved amino acid residue (dwf5-4). Position 3184, numbered relative to the start codon (position 3854 of the sequence shown in FIG. 7) in dwf5-3 was changed from cytidine to thymidine, replacing an Arg (CGA) with a premature stop codon (TGA) at amino acid residue 400 of FIG. 7. This premature stop codon resulted in deletion of most of the MCC. dwf5-4/le possessed a mutation changing guanosine at 1868, numbered relative to the start codon (position 2538 of the sequence shown in FIG. 7) to adenosine. This change resulted in the substitution of an acidic Asp (D) residue with an uncharged Asn (N) at amino acid position 257 of FIG. 7. The D was conserved in all seven peptide sequences compared (FIG. 5), suggesting a pivotal role for the D residue in this group of proteins. Another premature stop codon close to the C-terminal end, at nucleotide position 3881 of FIG. 7 (amino acid position 409) was found in dwf5-5. This mutation deleted only half of the MCC domain, but still caused a loss-of-function phenotype, suggesting that functionally important amino acid residues are present in the deleted half of the domain.

### EXAMPLE 8

### A Mutation Affecting mRNA Stability and Further RNA Analysis

dwf5-1 contained a single base deletion (adenosine at 3343, numbered relative to the start codon; position 4012 of the sequence shown in FIG. 7) near the stop codon (FIGS. 4A and 4B). The frameshift caused by this single base deletion is predicted to result in aberrant translation of 54 amino acids before the new stop codon. See the second row of amino acids corresponding to nucleotide positions 4013-4178 of FIG. 7. Hydrophathy analysis revealed that these newly added residues created an additional transmembrane domain. 3′ rapid amplification of cDNA ends (RACE) revealed that the size of mRNA from wild type and dwf5-1 was the same. This extended translation, consequently, resulted in shortening the 3′ untranslated region from 198 bp in wild type to 65 bp in dwf5-1. As such, the dwf5-1 phenotype may possibly be due to toxicity of this novel translation product or mRNA instability caused by translation of the 3′ UTR. To further investigate this issue steady state mRNA levels were examined in mutant and wild-type strains.

To determine the steady state levels of DWF5 mRNA in different tissues, total RNA was isolated from organs of 3-week-old Ws-2 wild-type plants. Tested tissues included the shoot Apex and unopened flowers (SAF), stems, mature siliques, pedicels, rosette leaves, roots, dark-grown seedlings, and callus. Roots were taken from 10-day-old seedlings grown on vertically oriented agar plates. For dark-grown tissues, seedlings were grown in the dark for 10 days. Root-derived callus was induced and grown on callus inducing media (Feldmann, 1992).

For expression levels in wild types and mutant alleles, plants of Ws-2, En-2 wild type, dwf5-1, dwf5-2, dwf5-3, dwf5-4, dwf5-5, dwf5-6, dwf7-1, dwf4-1, bri1-5 were grown for three to four weeks. Aerial parts of the plants were harvested and frozen with liquid nitrogen. RNA extraction with the RNEASY plant kit (Qiagen, Santa Clarita, Calif.) was performed following the manufacturer's directions. ³²P labeled DNA probes were made with the REDIPRIME II kit (Amersham Pharmacia, Piscataway, N.J.) using dwf5 cDNA and the tubulin α3 gene (TUA, Ludwig, et al., 1987) as templates.

FIGS. 6A and 6B present the results of RNA gel blot analysis of dwf5 transcripts. In FIG. 6A, RNA was isolated from two wild types, all of the dwf5 alleles, dwf7, dwf4, and bri1-1-5. dwf5 cDNA and an α3 tubulin gene (for loading control) were used as probes. Significantly decreased steady state mRNA levels were found in the dwf5-1 and dwf5-2 lanes.

In favor of the case for mRNA instability, the RNA gel blot analysis of dwf5 with total RNA isolated from different genotypes indicated that the steady state level of Δ⁷ reductase mRNA in dwf5-1 was significantly lower than that of wild type.

To examine how DWF5 expression is spatially controlled, RNA gel blot analysis was performed with total RNA isolated from different tissues including the shoot apex and unopened flowers (SAF), stems, mature siliques, pedicels, rosette leaves, roots, dark-grown seedlings, and callus (FIG. 6B).

In FIG. 6B, total RNA was isolated from the tissues indicated, and probed with dwf5 cDNA. dwf5 was strongly detected in the SAF and roots. As a loading control, the 28S rRNA region of the ethidium bromide-stained gel is shown.

The steady state level of mRNA was highest in the SAF and roots. Stems, callus, and rosette leaves showed moderate expression of the gene. Expression in siliques, dark-grown seedlings and pedicels was comparatively low among the tissues examined.

### EXAMPLE 9

### Molecular Complementation

Molecular complementation experiments were performed for several reasons. First, to prove unequivocally that a mutation in the S7R gene was responsible for the dwf5-1 phenotype. Second, because the human S7R gene was cloned using the Arabidopsis gene as a probe (Moebius, et al., 1998), it was of interest to test if the human gene could rescue the dwf5 phenotype. To this end, overexpression constructs were made using dwf5 and human cDNAs. In particular, the construct AOH5 includes the human cDNA sequence of GenBank Accession No. AF034544 driven by the CaMV 35S constitutive promoter. The backbone plasmid vectors for AOH5 were pART7 and pART27 (Gleave et al., 1992).

The AOH5 and AOD5 constructs were introduced into the Agrobacterium strain GV3101 by electroporation. The Agrobacterium strains harboring the constructs were introduced into dwf5-1 mutant plants by a floral dip method (Clough and Bent, 1998). Successful transformants were isolated by screening for kanamycin-resistant plants grown on agar-solidified plates containing 50 μg/ml kanamycin. Ectopic overexpression of the dwf5 cDNA (AOD5) in dwf5-1 plants completely converted the mutant to wild-type phenotype.

As compared to dwf5-1, the AOD5 plant was completely reverted to the wild-type phenotype, indicating that the single base deletion in the S7R gene is the cause of the mutant phenotype. Transgenic plants containing the human overexpression construct AOH5, while larger, were not rescued to wild type. Further, biochemical analysis of sterols in AOH5 plants showed that their levels were comparable to dwf5 controls.

Thus, novel dwf5 mutants, the DWF5 genomic sequence, as well as methods of using the same, are disclosed. Although preferred embodiments of the subject invention have been described in some detail, it is understood that obvious variations can be made without departing from the spirit and the scope of the invention as defined by the appended claims.

### REFERENCES

Altmann, T. (1998). A tale of dwarfs and drugs: brassinosteroids to the rescue. Trends Genet. 14, 490-495.

Altschul, S. F., Madden, T. L., Schaffer, A. A., Zhang, J., Zhang, Z., Miller W. and Lipman, D. J. (1997). Gapped BLAST and PSI-BLAST: a new generation of protein database search programs. Nucleic Acids Res. 25, 3389-3402.

Azpiroz, R., Wu, Y., LoCascio, J. C. and Feldmann, K. A. (1998). An Arabidopsis brassinosteroid-dependent mutant is blocked in cell elongation. Plant Cell, 10, 219-230.

Bach, T. J. and Benveniste, P. (1997). Cloning of cDNAs or genes encoding enzymes of sterol biosynthesis from plants and other eukaryotes: heterologous expression and complementation analysis of mutations for functional characterization. Prog. Lipid Res. 36, 197-226.

Barton, G. J. (1993). ALSCRIPT a tool to format multiple sequence alignments. Protein Eng. 6, 37-40.

Bell, C. J. and Ecker, J. R. (1994). Assignment of 30 microsatellite loci to the linkage map of Arabidopsis. Genomics, 19, 137-144.

Bishop, G., Harrison, K. and Jones, J. D. G. (1996). The Tomato Dwarf gene isolated by heterologous transposon tagging encodes the first member of a new cytochrome P450 family. Plant Cell, 8, 959-969.

Bishop, G. J., Nomura, T., Yokota, T., Harrison, K., Noguchi, T., Fujioka, S., Takatsuto, S., Jones, J. D. and Kamiya, Y. (1999). The tomato DWARF enzyme catalyses C-6 oxidation in brassinosteroid biosynthesis. Proc. Natl. Acad. Sci. USA, 96, 1761-1766.

Brendel, V., Bucher, P., Nourbakhsh, I. R., Blaisdell, B. E. and Karlin, S. (1992). Methods and algorithms for statistical analysis of protein sequences. Proc. Natl. Acad. Sci. USA, 89, 2002-2006.

Choe, S., Dilkes, B. P., Fujioka, S., Takatsuto, S., Sakurai, A. and Feldmann, K. A. (1998). The DWF4 gene of Arabidopsis encodes a cytochrome P450 that mediates multiple 22α-hydroxylation steps in brassinosteroid biosynthesis. Plant Cell, 10, 231-243.

Choe, S., Dilkes, B. P., Gregory, B. D., Ross, A. S., Yuan, H., Noguchi, T., Fujioka, S., Takatsuto, S., Tanaka, A., Yoshida, S., Tax, F. E. and Feldmann, K. A. (1999a). The Arabidopsis dwarf1 mutant is defective in the conversion of 24-methylenecholesterol to campesterol in brassinosteroid biosynthesis. Plant Physiol. 119, 897-907.

Choe, S., Noguchi, T., Fujioka, S., Takatsuto, S., Tissier, C. P., Gregory, B. D., Ross, A. S., Tanaka, A., Yoshida, S., Tax, F. E. and Feldmann, K. A. (1999b). The Arabidopsis dwf7/ste1 mutant is defective in the Δ⁷ sterol C-5 desaturation step leading to brassinosteroid biosynthesis. Plant Cell, 11, 207-221.

Clough, S. J. and Bent, A. F. (1998). Floral dip: a simplified method for Agrobacterium-mediated transformation of Arabidopsis thaliana. Plant J. 16, 735-743.

Clouse, S. D. and Sasse, J. M. (1998). Brassinosteroids: essential regulators of plant growth and development. Annu. Rev. Plant Physiol. Plant Mol. Biol. 49, 427-451.

Clouse, S. D., Langford, M. and McMorris, T. C. (1996). A brassinosteroid-insensitive mutant in Arabidopsis thaliana exhibits multiple defects in growth and development. Plant Physiol. 111, 671-678.

Clouse, S. D. and Feldmann, K. A. (1999). Molecular genetics of brassinosteroid action. In Brassinosteroids: steroidal plant hormones (Sakurai, A. Yokota, T. and Clouse, S. D., eds). Tokyo, Japan: Springer, pp. 163-190.

Ephritikhine, G., Pagant, S., Fujioka, S., Takatsuto, S., Lapous, D., Caboche, M.,

Kendrick, R. E. and Barbier-Brygoo, H. (1999). The sax1 mutation defines a new locus involved in the brassinosteroid biosynthesis pathway in Arabidopsis thaliana. Plant J. 18, 315-320.

Feldmann, K. A. (1992). In vitro culture of Arabidopsis thaliana. In Biotechnology in agriculture and forestry: high-tech and micropropagation IV (Y. P. S. Bajaj, ed). Berlin: Springer-Verlag, pp. 471-483.

Feldmann, K. A., Marks, M. D., Christianson, M. L. and Quatrano, R. S. (1989). A dwarf mutant of Arabidopsis generated by T-DNA insertion mutagenesis. Science, 243, 1351-1354.

Fitzky, B. U., Witsch-Baumgartner, M., Erdel, M., Lee, J. N., Paik, Y. K., Glossmann, H., Utermann, G. and Moebius, F. F. (1998). Mutations in the Δ⁷-sterol reductase gene in patients with the Smith-Lemli-Opitz syndrome. Proc. Natl. Acad. Sci. USA, 95, 8181-8186.

Fujioka, S., Li, J., Choi, Y. H., Seto, H., Takatsuto, S., Noguchi, T., Watanabe, T., Kuriyama, H., Yokota, T., Chory, J. and Sakurai, A. (1997). The Arabidopsis deetiolated2 mutant is blocked early in brassinosteroid biosynthesis. Plant Cell, 9, 1951-1962.

Gachotte, D., Meens, R. and Benveniste, P. (1995). An Arabidopsis mutant deficient in sterol biosynthesis: heterologous complementation by ERG3 encoding a Δ⁷-sterol-C-5-desaturase from yeast. Plant J. 8, 407-416.

Gachotte, D., Husselstein, T., Bard, M., Lacroute, F. and Benveniste, P. (1996). Isolation and characterization of an Arabidopsis thaliana cDNA encoding a Δ⁷-sterol-C-5-desaturase by functional complementation of a defective yeast mutant. Plant J. 9, 391-398.

Gant, T. M. and Wilson, K. L. (1997). Nuclear assembly. Annu. Rev. Cell Dev. Biol. 13, 669-695.

Gleave, A. P. (1992). A versatile binary vector system with a T-DNA organisational structure conducive to efficient integration of cloned DNA into the plant genome. Plant Mol. Biol. 20, 1203-1207.

Husselstein, T., Schaller, H., Gachotte, D. and Benveniste, P. (1999). Δ⁷-sterol-C5-desaturase: molecular characterization and functional expression of wild-type and mutant alleles. Plant Mol. Biol. 39, 891-906.

Klahre, U., Noguchi, T., Fujioka, S., Takatsuto, S., Yokota, T., Nomura, T., Yoshida, S. and Chua, N. H. (1998). The Arabidopsis DIMINUTO/DWARF1 gene encodes a protein involved in steroid synthesis Plant Cell, 10, 1677-1690.

Konieczny, A. and Ausubel, F. M. (1993). A procedure for mapping Arabidopsis mutations using co-dominant ecotype-specific PCR-based markers. Plant J. 4, 403-410.

Kranz, A. R. and Scheidemann, U. (1978). Review on the linkage groups of Arabidopsis thaliana (L.) Heynh. Arabid. Inf. Serv. 15, 31-34.

Krysan, P. J., Young, J. C., Tax, F. and Sussman, M. R. (1996). Identification of transferred DNA insertions within Arabidopsis genes involved in signal transduction and ion transport. Proc. Natl. Acad. Sci. USA, 93, 8145-8150.

Lecain, E., Chenivesse, X., Spagnoli, R. and Pompon, D. (1996). Cloning by metabolic interference in yeast and enzymatic characterization of Arabidopsis thaliana sterol Δ⁷-reductase. J. Biol. Chem. 271, 10866-10873.

Li, J. and Chory, J. (1997). A putative leucine-rich repeat receptor kinase involved in brassinosteroid signal transduction. Cell, 90, 929-938.

Li, J., Nagpal, P., Vatart, V., McMorris, T. C. and Chory, J. (1996). A role for brassinosteroids in light-dependent development of Arabidopsis. Science, 272, 398-401.

Li, J., Biswas, M. G., Chao, A., Russel, D. W. and Chory, J. (1997). Conservation of function between mammalian and plant steroid 5-reductases. Proc. Natl. Acad. Sci. USA, 94,3554-3559.

Liu, Y.-G., Mitsukawa, N., Oosumi, T. and Whittier, R. F. (1995). Efficient isolation and mapping of Arabidopsis thaliana T-DNA insert junctions by thermal asymmetric interlaced PCR. Plant J. 8, 457-463.

Ludwig, S. R., Oppenheimer, D. G., Silflow, C. D. and Snustad, D. P. (1987). Characterization of the tubulin gene family of Arabidopsis thaliana. Proc. Natl. Acad. Sci. USA, 84, 5833-5837.

McKelvie, A. D. (1962). A list of mutant genes in Arabidopsis thaliana (L.) Heynh. Radiat. Bot. 1, 233-241.

Moebius, F. F., Fitzky, B. U., Lee, J. N., Paik, Y. K. and Glossmann, H. (1998). Molecular cloning and expression of the human Δ⁷-sterol reductase. Proc. Natl. Acad. Sci. USA, 95, 1899-1902.

Noguchi, T., Fujioka, S., Takatsuto, S., Sakurai, A., Yoshida, S., Li J. and Chory, J. (1999a). Arabidopsis det2 is defective in the conversion of (24R)-24-methylcholest-4-en-3-one to (24R)-24-methyl-5-cholestan-3-one in brassinosteroid biosynthesis. Plant Physiol. 120, 833-839.

Noguchi, T., Fujioka, S., Choe, S., Takatsuto, S., Yoshida, S., Yuan, H., Feldmann, K. A. and Tax, F. E. (1999b). Brassinosteroid-insensitive (bri1) dwarf mutants of Arabidopsis accumulate brassinosteroids. Plant Physiol. 121, in press.

Nomura, T., Kitasaka, Y., Takatsuto, S., Reid, J. B., Fukami, M. and Yokota, T. (1999). Brassinosteroid/sterol synthesis and plant growth as affected by lka and lkb mutations of pea. Plant Physiol. 119, 1517-1526.

Salen, G., Shefer, S., Batta, A. K., Tint, G. S., Xu, G., Honda, A., Irons, M. and Elias, E. R. (1996). Abnormal cholesterol biosynthesis in the Smith-Lemli-Opitz syndrome. J. Lipid Res. 37, 1169-1180.

Sambrook, J., Fritsch, E. F. and Maniatis, T. (1989). Molecular cloning: A Laboratory Manual. Cold Spring Harbor, N.Y.: Cold Spring Harbor Laboratory Press.

Silve, S., Dupuy, P. H., Ferrara, P. and Loison, G. (1998). Human lamin B receptor exhibits sterol C14-reductase activity in Saccharomyces cerevisiae. Biochim. Biophys. Acta, 1392, 233-244.

Simpson, G. G. and Filipowicz, W. (1996). Splicing of precursors to mRNA in higher plants: mechanism, regulation and sub-nuclear organisation of the spliceosomal machinery. Plant Mol. Biol. 32, 1-41.

Smith, D. W., Lemli, L. and Opitz, J. M. (1964). A newly recognized syndrome of multiple congenital anomalies. J. Pediatr. 64, 210-217

Szekeres, M., Nemeth, K., Koncz-Kalman, Z., Mathur, J., Kauschmann, A., Altmann, T., Redei, G. P., Nagy, F., Schell, J. and Koncz, C. (1996). Brassinosteroids rescue the deficiency of CYP90, a cytochrome P450 controlling cell elongation and de-etiolation in Arabidopsis. Cell, 85, 171-182.

Takahashi, T., Gasch, A., Nishizawa N. and Chua, N.-H. (1995). The DIMINUTO gene of Arabidopsis is involved in regulating cell elongation. Genes Dev. 9, 97-107. Taton, M. and Rahier, A. (1991). Identification of 5,7-sterol-Δ⁷-reductase in higher plant microsomes. Biochem. Biophys. Res. Commun. 181, 465-473.

Wassif, C. A., Maslen, C., Kachilele-Linjewile, S., Lin, D., Linck, L. M., Connor, W. E., Steiner, R. D. and Porter, F. D. (1998). Mutations in the human sterol Δ⁷-reductase gene at 11q12-13 cause Smith-Lemli-Opitz syndrome. Am. J. Hum. Genet. 63, 55-62.

Waterham, H. R., Wijburg, F. A., Hennekam, R. C., Vreken, P., Poll-The, B. T., Dorland, L., Duran, M., Jira, P. E., Smeitink, J. A., Wevers, R. A. and Wanders. R. J., (1998). Smith-Lemli-Opitz syndrome is caused by mutations in the 7-dehydrocholesterol reductase gene. Am. J. Hum. Genet. 63, 329-338.

What is claimed is:

1. An isolated polynucleotide selected from the group consisting of (a) a dwf5-1 polynucleotide comprising the dwf5-1 nucleotide sequence depicted in FIG. 7; (b) a dwf5-2 polynucleotide comprising the dwf5-2 nucleotide sequence depicted in FIG. 7; (c) a dwf5-3 polynucleotide comprising the dwf5-3 nucleotide sequence depicted in FIG. 7; (d) a dwf5-4 polynucleotide comprising the dwf5-4 nucleotide sequence depicted in FIG. 7; (e) a dwf5-5 polynucleotide comprising the dwf5-5 nucleotide sequence depicted in FIG. 7; (f) a dwf5-6 polynucleotide comprising the dwf5-6 nucleotide sequence depicted in FIG. 7; (g) a DWF5 polynucleotide comprising the genomic DWF5 sequence depicted in FIG. 7; (h) a polynucleotide comprising a nucleotide sequence having at least about 50% sequence identity to the nucleotide sequence of (a), (b), (c), (d), (e), (f) or (g); (i) a fragment of (a), (b), (c), (d), (e), (f), (g) or (h) comprising at least about 15 contiguous nucleotides therefrom; and (j) complements or reverse complements of (a), (b), (c), (d), (e), (f), (g), (h) or (i).

2. The isolated polynucleotide of claim 1, wherein said polynucleotide imparts at least one dwf5 mutant phenotype when expressed in a plant.

3. The isolated polynucleotide of claim 1 comprising at least 30 contiguous nucleotides of (a), (b), (c), (d), (e), (f), (g) or (h).

4. An isolated polynucleotide selected from the group consisting of (a) a polynucleotide comprising a nucleotide sequence having at least about 50% sequence identity to the genomic DWF5 sequence depicted in FIG. 7; (b) a fragment of (a) comprising at least about 15 contiguous nucleotides therefrom; and (c) complements or reverse complements of (a) or (b).

5. The isolated polynucleotide of claim 4 comprising at least 30 contiguous nucleotides of (a) or (b).

6. An isolated polynucleotide selected from the group consisting of (a) a polynucleotide comprising the nucleotide sequence depicted at nucleotide positions 1-633 of FIG. 7; (b) a polynucleotide comprising the nucleotide sequence depicted at nucleotide positions 634-670 of FIG. 7; (c) a polynucleotide comprising the nucleotide sequence depicted at nucleotide positions 4045-4243 of FIG. 7; (d) a polynucleotide comprising an intron sequence as depicted in FIG. 7; (e) a polynucleotide comprising a nucleotide sequence having at least about 50% sequence identity to the nucleotide sequence of (a), (b), (c) or (d); (f) a fragment of (a), (b), (c), (d) or (e) comprising at least about 15 contiguous nucleotides therefrom; and (g) complements or reverse complements of (a), (b), (c), (d), (e) or (f).

7. The isolated polynucleotide of claim 6 comprising at least 30 contiguous nucleotides of (a), (b), (c), (d) or (e).

8. A recombinant vector comprising (i) the polynucleotide of any of claims 1-5; and (ii) control elements operably linked to said polynucleotide whereby a coding sequence within said polynucleotide can be transcribed and translated in a host cell.

9. A recombinant vector comprising: (i) a polynucleotide of any of claims 6 and 7 which includes a DWF5 control element; and (ii) a heterologous coding sequence operably linked to said polynucleotide.

10. A host cell comprising the recombinant vector of claim 8.

11. A host cell comprising the recombinant vector of claim 9.

12. A method of producing a recombinant polypeptide comprising:
(a) providing a host cell according to claim 10; and
(b) culturing said host cell under conditions whereby a recombinant polypeptide encoded by the coding sequence present in said recombinant vector is expressed.

13. A method of producing a recombinant polypeptide comprising:
(a) providing a host cell according to claim 11; and
(b) culturing said host cell under conditions whereby a recombinant polypeptide encoded by the coding sequence present in said recombinant vector is expressed.

14. A transgenic plant comprising the polynucleotide of claim 1.

15. A method of producing a transgenic plant comprising:
(a) introducing the polynucleotide of claim 1 into a plant cell to produce a transformed plant cell; and
(b) producing a transgenic plant from the transformed plant cell.

16. A method of producing a transgenic plant comprising:
(a) introducing the recombinant vector of claim 8 into a plant cell to produce a transformed plant cell; and
(b) producing a transgenic plant from the transformed plant cell.

17. A method of producing a transgenic plant comprising:
(a) introducing the recombinant vector of claim 9 into a plant cell to produce a transformed plant cell; and
(b) producing a transgenic plant from the transformed plant cell.

18. A method for producing a transgenic plant having an altered phenotype relative to the corresponding wild-type plant comprising:
(a) introducing the polynucleotide of claim 1 into a plant cell; and
(b) producing a transgenic plant from the plant cell, said transgenic plant having an altered phenotype relative to the corresponding wild-type plant.

19. The method of claim 18, wherein the phenotype is altered sterol Δ⁷ reductase activity.

20. The method of claim 18, wherein the polynucleotide is operably linked to a promoter selected from the group consisting of a tissue-specific promoter, an inducible promoter and a constitutive promoter.

21. The method of claim 18, wherein the polynucleotide is overexpressed.

22. The method of claim 18, wherein the polynucleotide inhibits expression of dwf5.

23. The method of claim 18, wherein at least first and second polynucleotides are introduced into the plant cell, said first and second polynucleotides operably linked to at least first and second tissue-specific promoters, wherein said first polynucleotide is overexpressed and said second polynucleotide inhibits expression of dwf5.

24. A method of modulating an endogenous DWF5 polypeptide in a transgenic plant comprising providing a polynucleotide according to claim 1.

25. The method of claim 24, wherein the polynucleotide is overexpressed.

26. The method of claim 24, wherein expression of the polynucleotide is inhibited.

27. A method for altering the biochemical activity of a cell comprising:
(a) introducing at least one polynucleotide according to claim 1 into the cell; and
(b) causing expression of said polynucleotide such that the biochemical activity of the cell is altered.

28. The method of claim 27, wherein the biochemical activity is selected from the group consisting of altered sterol Δ⁷ reductase activity and altered sterol composition.

29. The method of claim 27, wherein the polynucleotide is introduced into the cell ex vivo.

30. The method of claim 27, wherein the polynucleotide is introduced into the cell in vivo.

31. The method of claim 27, wherein more than one dwf5 polynucleotide is provided to the cell.

32. A method for regulating the cell cycle of a plant cell comprising:
(a) providing a polynucleotide according to claim 1 to a plant cell; and
(b) expressing the polynucleotide to provide a DWF5 polypeptide, wherein the DWF5 polypeptide is provided in amounts such that cell cycling is regulated.

33. The method of claim 32, wherein the plant cell is provided in vitro and is cultured under conditions suitable for expressing the DWF5 polypeptide.

34. The method of claim 32, wherein the polynucleotide is provided in vivo.

35. A method of modulating mRNA levels in a plant cell comprising:
(a) providing a plant cell; and
(b) introducing the recombinant vector of claim 9 into the plant cell to produce a transformed plant cell.

36. The method of claim 35, wherein the plant cell is from a plant tissue selected from the group consisting of the shoot apex and unopened flower (SAF), stem, mature silique, pedicel, rosette leaf, root, dark-grown seedling and callus.

37. The method of claim 35, wherein the recombinant vector is introduced into the plant cell in vitro.

38. The method of claim 35, wherein the recombinant vector is introduced into the plant cell in vivo.

39. A chimeric polypeptide comprising a first amino acid sequence of a DWF5 polypeptide and a second amino acid sequence of a heterologous polypeptide.
